(19) United States
(12) Patent Application Publication
(10) Pub. No.: US 2023/0102128 A1
(43) Pub. Date: Mar. 30, 2023

(54) PLANT DISEASE CONTROL COMPOSITION, AND PLANT DISEASE CONTROL METHOD

(71) Applicant: SUMITOMO CHEMICAL COMPANY, LIMITED, Tokyo (JP)

(72) Inventors: Hidekatsu TOBITA, Takarazuka-shi, Hyogo (JP); Yoshihiko NOKURA, Takarazuka-shi, Hyogo (JP); Takaaki NAKANO, Kasai-shi, Hyogo (JP)

(73) Assignee: SUMITOMO CHEMICAL COMPANY, LIMITED, Tokyo (JP)

(21) Appl. No.: 17/796,099

(22) PCT Filed: Jan. 29, 2021
(86) PCT No.: PCT/JP2021/003319

§ 371 Date: Jul. 28, 2022

(30) Foreign Application Priority Data
Jan. 31, 2020 (JP) 2020-015190

(51) Int. Cl. A01N 43/40; A01N 37/36

(52) U.S. Cl. CPC A01N 43/40; A01N 37/36

(57) ABSTRACT

The present invention provides a composition having an excellent control efficacy against plant diseases, and a method for controlling plant diseases using the same composition, and the composition comprises a compound represented by formula (I) [wherein each symbol represents the definition described in the specification], and one or more compounds selected from a mitochondrial electron transport chain complex III inhibitor, a mitochondrial electron transport chain complex II inhibitor, and a sterol biosynthesis inhibitor.

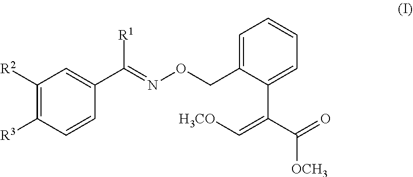

### TECHNICAL FIELD

This application claims priority to and the benefit of Japanese Patent Application Nos. 2020-015190 filed Jan. 31, 2020, the entire contents of which are incorporated herein by reference.

The present invention relates to a composition for controlling plant diseases and a method for controlling plant diseases.

### BACKGROUND ART

Hitherto, many compounds have been known as an active ingredient for a composition for controlling plant diseases (see Non-Patent Literature 1).

### CITATION LIST

### Non-Patent Literature

Non-Patent Literature 1: The Pesticide Manual-18th Edition (published by BCPC); ISBN 978-1-9998966-1-4

### SUMMARY OF THE INVENTION

### Problems to be Solved by Invention

An object of the present invention is to provide a composition for controlling plant diseases having excellent efficacy for controlling plant diseases, and a method for controlling plant diseases using the same composition.

### Means to Solve Problems

The present inventors have intensively studied to find out a composition having an excellent efficacy for controlling a plant disease, and a method for controlling a plant disease using the same composition, and as a result, found that a composition comprising a compound represented by the following formula (I) and one or more compounds selected from Group (B) has an excellent efficacy for controlling plant diseases.

That is, the present invention encompasses the followings.

[1] A composition for controlling a plant disease comprising a compound represented by the following formula (I) and one or more compounds selected from Group (B).

a formula (I):

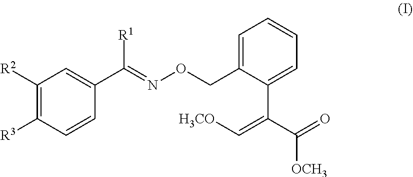

[wherein

R¹ represents a C1-C3 alkyl group,

R² and R³ are identical to or different from each other and represent a hydrogen atom, a halogen atom, a C1-C3 alkyl group optionally having one or more halogen atoms, or a C1-C3 alkoxy group optionally having one or more halogen atoms, or

R² and R³ may combine with each other to form —CH₂CH₂CH₂—, or —CH₂CH₂CH₂CH₂—],

Group (B): a group consisting of the following sub-groups (B-1), (B-2) and (B-3),
Sub-group (B-1): a mitochondrial electron transport chain complex III inhibitor

a group consisting of picoxystrobin, pyraclostrobin, metyltetraprole, fenpicoxamid, and florylpicoxamid.

Sub-group (B-2): a mitochondrial electron transport chain complex II inhibitor

a group consisting of fluxapyroxad, benzovindiflupyr, fluindapyr, pydiflumetofen, 3-(difluoromethyl)-N-(2,3-dihydro-1,1,3-trimethyl-1H-inden-4-yl)-1-methyl-1H-pyrazole-4-carboxamide, a compound represented by formula (1), a compound represented by formula (2), and a compound represented by formula (3).

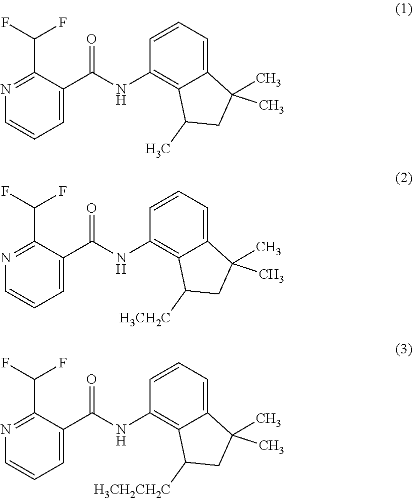

Sub-group (B-3): a sterol biosynthesis inhibitor mefentrifluconazole.
[2] The composition for controlling a plant disease according to [1], wherein the compound represented by formula (I) is a compound of formula (I) wherein R² represents a hydrogen atom, R³ represents a C1-C3 alkyl group optionally having one or more halogen atoms, a C1-C3 alkoxy group optionally having one or more halogen atoms, or a halogen atom.
[3] The composition for controlling a plant disease according to [1], wherein the compound represented by formula (I) is a compound of formula (I) wherein R³ represents a hydrogen atom, R² represents a C1-C3 alkyl group optionally having one or more halogen atoms, a C1-C3 alkoxy group optionally having one or more halogen atoms, or a halogen atom.
[4] The composition for controlling a plant disease according to [1], wherein the compound represented by formula (I) is
methyl(2E)-2-(2-{[({(1E)-1-[5,6,7,8-tetrahydronaphthalen yl]ethylidene}amino)oxy]methyl}phenyl)-3-methoxy-2-propenoate;
methyl(2E)-2-(2-{[({(1E)-1-[2,3-dihydro-1H-inden-5-yl]ethylidene}amino)oxy]methyl}phenyl)-3-methoxy-2-propenoate;
methyl(2E)-2-(2-{[({(1E)-1-[3-chlorophenyl]ethylidene}amino)oxy]methyl}phenyl)-3-methoxy-2-propenoate;
methyl(2E)-2-(2-{[({(1E)-1-[4-chlorophenyl]ethylidene}amino)oxy]methyl}phenyl)-3-methoxy-2-propenoate;
methyl(2E)-2-(2-{[({(1E)-1-[3-(trifluoromethyl)phenyl]ethylidene}amino)oxy]methyl}phenyl)-3-methoxy-2-propenoate;
methyl(2E)-2-{2-[({[(1E)-1-(4-methylphenyl)propylidene]amino}oxy)methyl]phenyl}-3-methoxy-2-propenoate; or
methyl(2E)-2-(2-{[({(1E)-1-[4-(trifluoromethyl)phenyl]propylidene}amino)oxy]methyl}phenyl)-3-methoxy-2-propenoate.
[5] The composition for controlling a plant disease according to any one of [1] to [4], wherein

in Group (B),

Sub-group (B-1) is a group selected from a group consisting of metyltetraprole, fenpicoxamid, and florylpicoxamid,

Sub-group (B-2) is selected from a group consisting of pydiflumetofen, 3-(difluoromethyl)-N-(2,3-dihydro-1,1,3-trimethyl-1H-inden-4-yl)-1-methyl-1H-pyrazole-4-carboxamide, a compound represented by formula (1), a compound represented by formula (2), and a compound represented by formula (3):

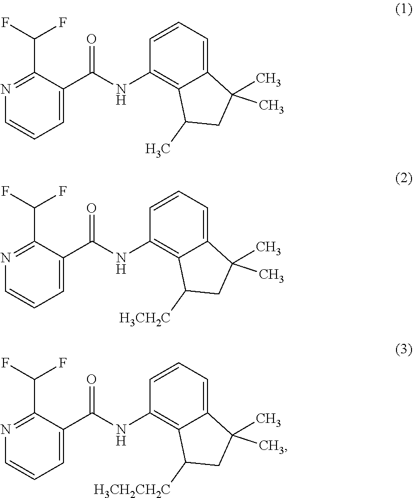

and

Sub-group (B-3) is mefentrifluconazole.

[6] The composition for controlling a plant disease according to any one of [1] to [5], wherein a weight ratio of the compound represented by formula (I) and one or more of the compounds selected from Group (B) is within a range of 1:0.01 to 1:100.
[7] The composition for controlling a plant disease according to any one of [1] to [5], wherein a weight ratio of the compound represented by formula (I) and one or more of the compounds selected from Group (B) is within a range of 1:0.1 to 1:10.
[8] A method for controlling a plant disease which comprises applying an effective amount of the composition for controlling a plant disease according to any one of [1] to [7] to a plant or soil where a plant grows.
[9] Use of the composition for controlling a plant disease according to any one of [1] to [7] for controlling a plant disease.
[10] The composition for controlling a plant disease according to any one of [1] to [4], wherein the Group (B) is a sub-group (B-1).
[11] The composition for controlling a plant disease according to [10], wherein the sub-group (B-1) is selected from the group consisting of metyltetraprole, fenpicoxamid, and florylpicoxamid.
[12] The composition for controlling a plant disease according to [10] or [11], wherein a weight ratio of the compound represented by formula (I) and one or more of the compounds selected from the Group (B) is within a range of 1:0.01 to 1:100 or 1:0.1 to 1:10.
[13] The composition for controlling a plant disease according to any one of [1] to [4], wherein the Group (B) is the sub-group (B-2).
[14] The composition for controlling a plant disease according to [13], wherein the sub-group (B-2) is selected from the group consisting of pydiflumetofen, 3-(difluoromethyl)-N-(2,3-dihydro-1,1,3-trimethyl-1H-inden yl)-1-methyl-1H-pyrazole-4-carboxamide, a compound represented by formula (1), a compound represented by formula (2), and a compound represented by formula (3).

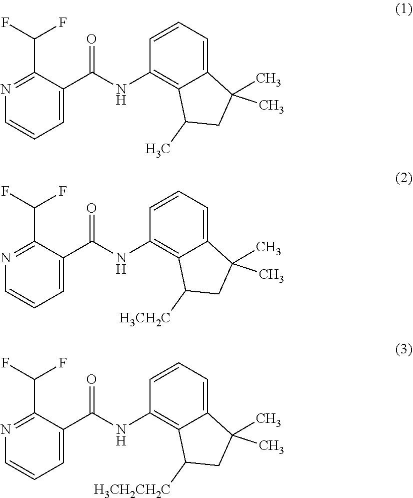

[15] The composition for controlling a plant disease according to [13] or [14], wherein a weight ratio of the compound represented by formula (I) and one or more of the compounds selected from Group (B) is within a range of 1:0.01 to 1:100, or 1:0.1 to 1:10.
[16] The composition according to any one of [1] to [4], wherein the Group (B) is the sub-group (B-3).
[17] The composition for controlling a plant disease according to [16], wherein a weight ratio of the compound represented by formula (I) and one or more of the compounds selected from Group (B) is within a range of 1:0.01 to 1:100, or 1:0.1 to 1:10.

### Effect of Invention

The composition for controlling plant diseases of the present invention can control plant diseases.

### MODE FOR CARRYING OUT THE INVENTION

The composition for controlling plant diseases of the present invention (hereinafter, referred to as “Present composition” or “Composition of the present invention”) comprises a compound represented by the above-mentioned formula (I) (hereinafter, referred to as “Present compound A”) and one or more compounds selected from Group (B) (hereinafter, referred to as “Present compound B”).

Firstly, the substituent(s) as described herein is/are explained.

The term “halogen atom” represents fluorine atom, chlorine atom, bromine atom, or iodine atom.

When the substituents have two or more halogen atoms, these halogen atoms may be identical to or different from each other.

The expression of “CX-CY” as used herein represents that the number of carbon atom is from X to Y. For example, the expression of “C1-C3” represents that the number of carbon atom is from 1 to 3.

Examples of “alkyl group” include methyl group, ethyl group, propyl group, and isopropyl group.

In the compound represented by formula (I), examples of the compound wherein “R² and R³ may combine with each other to form —CH₂CH₂CH₂—, or —CH₂CH₂CH₂CH₂—” include the compound represented by the following formula (II), and the compound represented by the following formula (III).

A compound represented by formula (II):

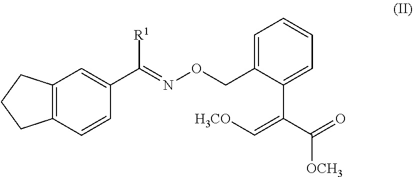

[wherein the symbols are the same as defined above.]

A compound represented by formula (III):

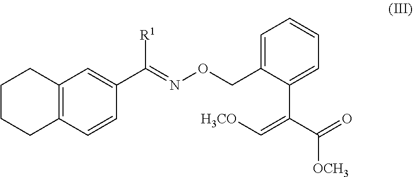

[wherein the symbols are the same as defined above.]

The present compound A may be existed as one or more stereoisomers. Examples of the stereoisomer include enantiomer, diastereoisomer, and geometric isomer. Each stereoisomer, and stereoisomer mixture(s) in an arbitrary ratio thereof are included in the present compound A.

Examples of the embodiment of the present compound A include the following compounds.

### Embodiment 1

The present compound A wherein R¹ represents a methyl group, or an ethyl group.

### Embodiment 2

The present compound A wherein R¹ represents a methyl group.

### Embodiment 3

The compound according to the embodiment 2 wherein R² and R³ are identical to or different from each other and represent a hydrogen atom, a halogen atom, a methyl group which may be optionally substituted with one or more halogen atoms, or a methoxy group which may be optionally substituted with one or more halogen atoms.

### Embodiment 4

The compound according to the embodiment 2 wherein R² and R³ are identical to or different from each other and represent a hydrogen atom, a chlorine atom, a trifluormethyl group, or a trifluoromethoxy group.

### Embodiment 5

The compound according to the embodiment 2 wherein R² represents a hydrogen atom, and R³ represents a chlorine atom, a trifluoromethyl group, or a trifluoromethoxy group.

### Embodiment 6

The compound according to the embodiment 2 wherein R³ represents a hydrogen atom, and R² represents a chlorine atom, a trifluoromethyl group, or a trifluoromethoxy group.

### Embodiment 7

A compound represented by formula (II):

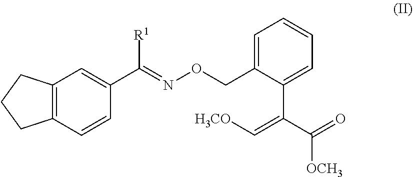

wherein R¹ represents a methyl group or an ethyl group.

### Embodiment 8

The compound according to the embodiment 7 wherein R¹ represents a methyl group.

### Embodiment 9

A compound represented by formula (III):

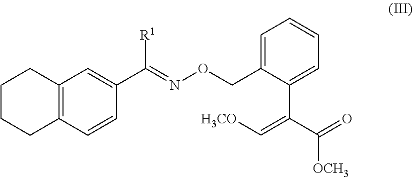

wherein R¹ represents a methyl group or an ethyl group.

### Embodiment 10

The compound according to the embodiment 9 wherein R¹ represents a methyl group.

Examples of the embodiments of the present composition include the following compositions.

### Embodiment 11

The composition wherein a weight ratio of the compound represented by formula (I) according to the above [2] and one or more of the compounds selected from Group (B) is within a range of 1:0.01 to 1:100.

### Embodiment 12

The composition according to the above [3] wherein a weight ratio of the compound represented by formula (I) and one or more of the compounds selected from Group (B) is within a range of 1:0.01 to 1:100.

### Embodiment 13

The composition according to the above [4] wherein a weight ratio of the compound represented by formula (I) and one or more of the compounds selected from Group (B) is within a range of 1:0.01 to 1:100.

### Embodiment 14

The composition according to the above [2] wherein a weight ratio of the compound represented by formula (I) and one or more of the compounds selected from Group (B) is within a range of 1:0.1 to 1:10.

### Embodiment 15

The composition according to the above [3] wherein a weight ratio of the compound represented by formula (I) and one or more of the compounds selected from Group (B) is within a range of 1:0.1 to 1:10.

### Embodiment 16

The composition according to the above [4] wherein a weight ratio of the compound represented by formula (I) and one or more of the compounds selected from Group (B) is within a range of 1:0.1 to 1:10.

Next, a process for preparing the present compound A is described.

The present compound A can be prepared according to a similar method to those described in EP patent No. 0585751 B1 and WO 1990/07493 A1 and so on. Also the compound also can prepared by the below-mentioned preparation method.

### Process A

The present compound A can be prepared by reacting a compound represented by formula (M1) (hereinafter, referred to as “Compound (M-1)”) with a compound represented by formula (M2) (hereinafter, referred to as “Compound (M-2)”) in the presence of a base.

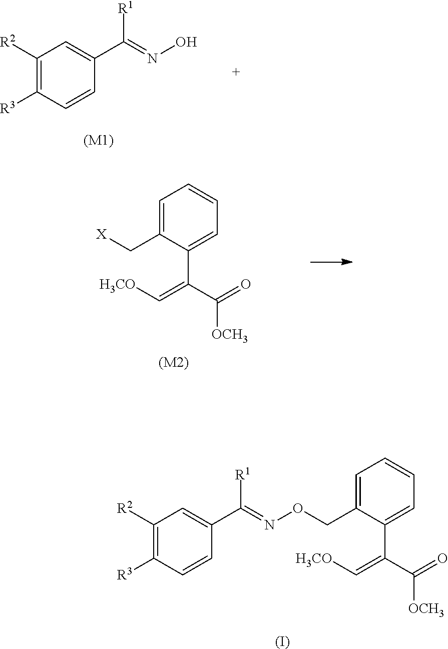

[wherein X represents a leaving group such as a chlorine atom, a bromine atom, an iodine atom, a methansulfonyloxy group, a trifluoromethanesulfonyloxy group, and the other symbols are the same as defined above.]

The reaction is usually conducted in a solvent. Examples of the solvents to be used in the reaction include hydrocarbon solvents such as heptane, toluene, and xylene; ethers such as tetrahydrofuran, and methyl tert-butyl ether; and amides such as N,N-dimethylformamide (hereinafter, referred to as DMF); esters such as ethyl acetate; sulfoxides such as dimethyl sulfoxide (hereinafter, referred to as DMSO); ketones such as acetone; nitriles such as acetonitrile; water; and mixed solvents of these two or more solvents.

Examples of the base to be used in the reaction include organic bases such as triethyl amine and pyridine; alkali metal carbonates such as sodium carbonate and potassium carbonate; alkali metal hydroxides such as sodium hydroxide and potassium hydroxide; and sodium hydride

In the reaction, the compound (M2) is usually used within a range of 1 to 10 molar ratio(s), and the base is usually used within a range of 1 to 10 molar ratio(s), as opposed to 1 mole of the compound (M1).

The reaction temperature is usually within a range of 0 to 150° C. The reaction period of the reaction is usually within a range of 0.1 to 24 hours.

In the reaction, sodium iodide, and tetrabutylammonium iodide may be added as needed, and these compounds may be used within a range of 0.001 to 1.2 molar ratios as opposed to 1 mole of the compound (M1).

When the reaction is completed, the reaction mixtures are extracted with organic solvent(s), and the organic layers are worked up (for example, drying and concentration) to isolate the present compound (A).

The compound (M1) and the compound (M2) are publicly known, or can be prepared according to a similar method to the publicly known method.

Next, the present compound B is described.

Examples of the present compound B include one or more compounds selected from the below-mentioned Sub-group (B-1), Sub-group (B-2) and Sub-group (B-3).

Sub-group (B-1): a mitochondrial electron transport chain complex III inhibitor

a group consisting of picoxystrobin, pyraclostrobin, metyltetraprole, fenpicoxamid, and florylpicoxamid.

Sub-group (B-2): a mitochondrial electron transport chain complex II inhibitor

a group consisting of fluxapyroxad, benzovindiflupyr, fluindapyr, pydiflumetofen, 3-(difluoromethyl)-N-(2,3-dihydro-1,1,3-trimethyl-1H-inden-4-yl)-1-methyl-1H-pyrazole-4-carboxamide, a compound represented by formula (1), a compound represented by formula (2), and a compound represented by formula (3).

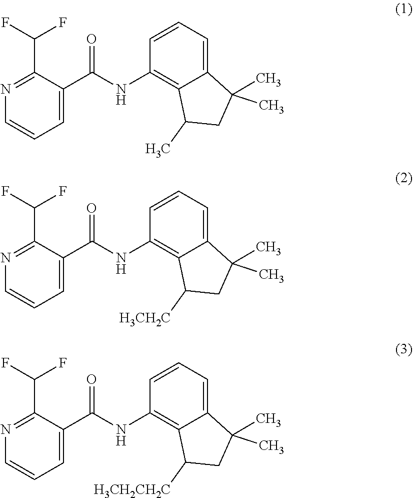

Sub-group (B-3): a sterol biosynthesis inhibitor mefentrifluconazole.

Picoxystrobin, pyraclostrobin, metyltetraprole, fenpicoxamid, florylpicoxamid, fluxapyroxad, benzovindiflupyr, fluindapyr, pydiflumetofen, 3-(difluoromethyl)-N-(2,3-dihydro-1,1,3-trimethyl-1H-inden-4-yl)-1-methyl-1H-pyrazole-4-carboxamide, and mefentrifluconazole are all publicly known compounds. These compounds are described, for example, at pages 904, 974, 789, 466, 498, 558, 97, 515, 969, 652, or 728 in The Pesticide Manual 18th Edition (published by BCPC), ISBN, 978-1-99998966-1-4. These compounds can be obtained from commercially available formulations, or can be obtained by preparing according to a publicly known method.

The compound represented by formula (1), the compound represented by formula (2), and the compound represented by formula (3), which are used in the present invention, are all publicly known compounds, and for example, are compounds described in WO 2014/095675 A1, and can be prepared by the process described in the publication.

In the composition of the present invention, a weight ratio of the present compound A and the present compound B is usually within a range of 1:0.01 to 1:100, preferably 1:0.1 to 1:10.

Though the composition for controlling plant diseases of the present invention may be mere mixtures of the present compound A and the present compound B, the composition of the present invention is usually mixed with the present compound A, and the present compound B, with solid carrier(s), liquid carrier(s), oils and/or surfactant(s), and the others, and if necessary, added by the other auxiliary agents for formulation, to formulate into emulsifiable concentrates, oil solutions, dust formulations, granules, wettable powders, wettable dispersible granules, flowables, dry flowables, microcapsules and the others. In these formulations, the total amount of the present compound A and the present compound B is within a range of usually 0.1 to 100% by weight, preferably 0.2 to 90% by weight, and more preferably 1 to 80% by weight.

Examples of the solid carrier include fine powders or granules of clays (for example, kaolin clay, diatomaceous earth, bentonite, or acid white clay), dry silica, wet silica, hydrated silica, talcs, ceramics, other inorganic minerals (for example, sericite, quartz, sulfur, active carbon, or calcium carbonate); chemical fertilizers (for example, ammonium sulfate, ammonium phosphate, ammonium nitrate, urea, or ammonium chloride); as well as synthetic resins (for example, polyester resins such as polypropylene, polyacrylonitrile, polymethyl methacrylate or polyethylene terephthalate; nylon resins (for example, nylon-6, nylon-11, or nylon-66); polyamide resins; polyvinyl chloride, polyvinylidene chloride, vinyl chloride-propylene copolymers, and the others).

Examples of the above-mentioned liquid carriers include water; alcohols (for example, methanol, ethanol, isopropyl alcohol, butanol, hexanol, benzyl alcohol, ethylene glycol, propylene glycol or phenoxy ethanol); ketones (for Example, acetone, methyl ethyl ketone or cyclohexanone); aromatic hydrocarbons (for example, toluene, xylene, ethyl benzene, dodecyl benzene, phenyl xylyl ethane or methylnaphthalene); aliphatic hydrocarbons (for example, hexane, cyclohexane, kerosene or light oil); esters (for example, ethyl acetate, butyl acetate, isopropyl myristate, ethyl oleate, diisopropyl adipate, diisobutyl adipate or propylene glycol monomethyl ether acetate); nitriles (for Example, acetonitrile or isobutyronitrile); ethers (for example, diisopropyl ether, 1,4-dioxane, ethylene glycol dimethyl ether, diethyleneglycol dimethyl ether, diethylene glycol monomethyl ether, propylene glycol monomethyl ether, dipropylene glycol monomethyl ether or 3-methoxy-3-methyl-1-butanol); amides (for example, DMF or N,N-dimethylacetamide); sulfoxides (for example, DMSO); propylene carbonate; and vegetable oils (for example, soybean oil or cottonseed oil).

Examples of the surfactants include nonionic surfactants such as polyoxyethylenated alkyl ethers, polyoxyethylenated alkyl aryl ethers, and polyethylene glycol fatty acid esters; and anionic surfactants such as alkyl sulfonates, alkylbenzene sulfonates and alkyl sulfates. Specific examples thereof include Nimbus (registered trademark), Assist (registered trademark), Aureo (registered trademark), Iharol (registered trademark), Silwet L-77 (registered trademark), BreakThru (registered trademark), SundanceII (registered trademark), Induce (registered trademark), Penetrator (registered trademark), AgriDex (registered trademark), Lutensol A8 (registered trademark), NP-7 (registered trademark), Triton (registered trademark), Nufilm (registered trademark), Emulgator NP7 (registered trademark), Emulad (registered trademark), TRITON X 45 (registered trademark), AGRAL 90 (registered trademark), AGROTIN (registered trademark), ARPON (registered trademark), EnSpray N (registered trademark), and BANOLE (registered trademark), and the others.

Examples of the other auxiliary agents for formulation include a binder, a dispersant, a colorant, and a stabilizer. Specific examples thereof include casein, gelatin, polysaccharides (for example, starch, gum arabic, cellulose derivatives and alginic acid), lignin derivatives, bentonite, water-soluble synthetic polymers (for example, polyvinyl alcohol, polyvinyl pyrrolidone and polyacrylic acids), acidic isopropyl phosphate, 2,6-di-tert-butyl-4-methylphenol, and BHA (that is, a mixture of 2-tert-butyl-4-methoxyphenol and 3-tert-butyl-4-methoxyphenol.

The composition of the present invention may be also prepared by formulating the present compound A and the present compound B respectively by each of the above-mentioned methods, and as needed by diluting the resulting formulations with water, and then mixing the respective formulation or the respective dilution thereof.

The composition of the present invention may further contain one or more kinds of other fungicide and/or pesticide.

The composition of the present invention can control plant diseases which are caused by phytopathogenic microorganism such as fungi, Oomycete, Phytomyxea, and bacteria and the others. Examples of the fungi include Ascomycota, Basidiomycota, Blasocladiomycota, Chytridiomycota, Mucoromycota, and Olpidiomycota. Specific examples thereof include the followings. The descriptions in a parenthesis indicates an academic name of phytopathogenic microorganism that causes each of the disease.

### Rice Diseases:

blast (Pyricularia oryzae), brown spot (Cochliobolus miyabeanus), sheath blight (Rhizoctonia solani), bakanae disease (Gibberella fujikuroi), downy mildew (Sclerophthora macrospora), false blast and head blight (Epicoccum nigrum), and seedling blight (Trichoderma viride, Rhizopus oryzae);

### Wheat Diseases:

powdery mildew (Blumeria graminis), Fusarium blight (Fusarium graminearum, Fusarium avenaceum, Fusarium culmorum, Microdochium nivale), stripe rust (Puccinia striiformis), stem rust (Puccinia graminis), leaf rust (Puccinia recondita), snow mould (Microdochium nivale, Microdochium majus), Typhula snow blight (Typhula incarnata, Typhula ishikariensis), loose smut (Ustilago tritici), stinking smut (Tilletia caries, Tilletia controversa), eyespot (Pseudocercosporella herpotrichoides), leaf blotch (Septoria tritici), glume blotch (Stagonospora nodorum), tan spot (Pyrenophora tritici-repentis), Rhizoctonia seeding blight (Rhizoctonia solani), take-all disease (Gaeumannomyces graminis), and blast (Pyricularia graminis-tritici);

### Barley Diseases:

powdery mildew (Blumeria graminis), Fusarium head blight (Fusarium graminearum, Fusarium avenaceum, Fusarium culmorum, Microdochium nivale), stripe rust (Puccinia striiformis), stem rust (Puccinia graminis), dwarf leaf rust (Puccinia hordei), loose smut (Ustilago nuda), scald (Rhynchosporium secalis), net blotch (Pyrenophora teres), spot blotch (Cochliobolus sativus), stripe (Pyrenophora graminea), Ramularia disease (Ramularia collo-cygni), and Rhizoctonia seeding blight (Rhizoctonia solani);

### Corn Diseases:

rust (Puccinia sorghi), southern rust (Puccinia polysora), northern leaf blight (Setosphaeria turcica), tropical rust (Physopella zeae), southern leaf blight (Cochliobolus heterostrophus), anthracnose (Colletotrichum graminicola), gray leaf spot (Cercospora zeae-maydis), eyespot (Kabatiella zeae), Phaeosphaeria leaf spot (Phaeosphaeria maydis), Diplodia rot (Stenocarpella maydis, Stenocarpella macrospora),
stalk rot (Fusarium graminearum, Fusarium verticilioides, Colletotrichum graminicola),
smut (Ustilago maydis), and Physoderma brown spot and Physoderma stalk rot (Physoderma maydis);

### Cotton Diseases:

anthracnose (Colletotrichum gossypii), grey mildew (Ramularia areola), Alternaria leaf spot (Alternaria macrospora, Alternaria gossypii), and black root rot (Thielaviopsis basicola);

### Coffee Diseases:

rust (Hemileia vastatrix), and leaf spot (Cercospora coffeicola);

### Rape Seed Diseases:

Sclerotinia rot (Sclerotinia sclerotiorum), gray leaf spot (Alternaria brassicae), root rot (Phoma lingam), and light leaf spot (Pyrenopeziza brassicae);

### Sugar Cane Diseases:

rust (Puccinia melanocephela, Puccinia kuehnii), and smut (Ustilago scitaminea);

### Sunflower Diseases:

rust (Puccinia helianthi), and downy mildew (Plasmopara halstedii);

### Citrus Diseases:

melanose (Diaporthe citri), scab (Elsinoe fawcetti), green mold (Penicillium digitatum), blue mold (Penicillium italicum), Phytophthora rot (Phytophthora parasitica, Phytophthora citrophthora), and Aspergillus rot (Aspergillus niger);

### Apple Diseases:

blossom blight (Monilinia mali), Valsa canker (Valsa ceratosperma), powdery mildew (Podosphaera leucotricha), Alternaria leaf spot (Alternaria alternata apple pathotype), scab (Venturia inaequalis), bitter rot (Glomerella cingulata, Colletotrichum acutatum),
blotch (Diplocarpon mali), ring rot (Botryosphaeria berengeriana), crown rot (Phytophtora cactorum), and rust (Gymnosporangium juniperi-virginianae, Gymnosporangium yamadae);

### Pear Diseases:

scab (Venturia nashicola, Venturia pirina), black spot (Alternaria alternata Japanese pear pathotype), and rust (Gymnosporangium haraeanum);

### Peach Diseases:

brown rot (Monilinia fructicola), scab (Cladosporium carpophilum), Phomopsis rot (Phomopsis sp.), and leaf curl (Taphrina deformans);

### Grapes Diseases:

anthracnose (Elsinoe ampelina), ripe rot (Glomerella cingulata, Colletotrichum acutatum), powdery mildew (Uncinula necator), rust (Phakopsora ampelopsidis), black rot (Guignardia bidwellii), and downy mildew (Plasmopara viticola);

### Japanese Persimmon Diseases:

anthracnose (Gloeosporium kaki, Colletotrichum acutatum), and leaf spot (Cercospora kaki, Mycosphaerella nawae);

### Diseases of Gourd Family:

anthracnose (Colletotrichum lagenarium), powdery mildew (Sphaerotheca fuliginea), gummy stem blight (Didymella bryoniae), Corynespora leaf spot (Corynespora cassiicola), Fusarium wilt (Fusarium oxysporum), downy mildew (Pseudoperonospora cubensis), Phytophthora rot (Phytophthora capsici), and damping-off (Pythium sp.);

### Tomato Diseases:

early blight (Alternaria solani), leaf mold (Cladosporium fulvum), Cercospora leaf mold (Pseudocercospora fuligena), late blight (Phytophthora infestans), and powdery mildew (Leveillula taurica);

### Eggplant Diseases:

brown spot (Phomopsis vexans), and powdery mildew (Erysiphe cichoracearum);

### Cruciferous Vegetables Diseases:

Alternaria leaf spot (Alternaria japonica), white spot (Cercosporella brassicae), clubroot (Plasmodiophora brassicae), downy mildew (Peronospora parasitica), and white rust (Albugo candida);

### Welsh Onion Disease:

rust (Puccinia allii);

### Soybean Diseases:

purple stain (Cercospora kikuchii), Sphaceloma scab (Elsinoe glycines), pod and stem blight (Diaporthe phaseolorum var. sojae), rust (Phakopsora pachyrhizi), target spot (Corynespora cassiicola), anthracnose (Colletotrichum glycines, Colletotrichum truncatum), Rhizoctonia rot (Rhizoctonia solani), Septoria brown spot (Septoria glycines), Cercospora leaf spot (Cercospora sojina), stem rot (Sclerotinia sclerotiorum), powdery mildew (Microsphaera diffusa), Phytophthora stem and root rot (Phytophthora sojae), downy mildew (Peronospora manshurica), sudden death syndrome (Fusarium virguliforme), red crown rot (Calonectria ilicicola), and Diaporthe/Phomopsis complex (Diaporthe longicolla);

### Kidney Bean Diseases:

stem rot (Sclerotinia sclerotiorum), rust (Uromyces appendiculatus), angular leaf spot (Phaeoisariopsis griseola), and anthracnose (Colletotrichum lindemuthianum), and Fusarium root-rot (Fusarium solani);

### Peanut Diseases:

leaf spot (Cercospora personata), brown leaf spot (Cercospora arachidicola), southern blight (Sclerotium rolfsii), and Cylindrocladium black rot (Calonectria ilicicola);

### Garden Pea Disease:

powdery mildew (Erysiphe pisi), and root rot (Fusarium solani);

### Potato Diseases:

early blight (Alternaria solani), late blight (Phytophthora infestans), Pink rot (Phytophthora erythroseptica), powdery scab (Spongospora subterranea f. sp. subterranea), Verticillium wilt (Verticillium albo-atrum, Verticillium dahliae, Verticillium nigrescens), dry rot (Fusarium solani), and potato wart (Synchytrium endobioticum);

### Strawberry Disease:

powdery mildew (Sphaerotheca humuli);

### Tea Diseases:

net blister blight (Exobasidium reticulatum), white scab (Elsinoe leucospila), gray blight (Pestalotiopsis sp.), and anthracnose (Colletotrichum theae-sinensis);

### Tobacco Diseases:

brown spot (Alternaria longipes), anthracnose (Colletotrichum tabacum), blue mold (Peronospora tabacina), and black shank (Phytophthora nicotianae);

### Sugar Beet Diseases:

Cercospora leaf spot (Cercospora beticola), leaf blight (Thanatephorus cucumeris), root rot (Thanatephorus cucumeris), and Aphanomyces root rot (Aphanomyces cochlioides), and rust (Uromyces betae);

### Rose Diseases:

black spot (Diplocarpon rosae), and powdery mildew (Sphaerotheca pannosa);

### Chrysanthemum Diseases:

leaf blight (Septoria chrysanthemi-indici), and white rust (Puccinia horiana);

### Onion Diseases:

Botrytis leaf blight (Botrytis cinerea, Botrytis byssoidea, Botrytis squamosa), gray-mold neck rot (Botrytis allii), and small sclerotial neck rot (Botrytis squamosa);

### Various Crops Diseases:

Botrytis rot (Botrytis cinerea), Sclerotinia rot (Sclerotinia sclerotiorum), seedling blight (Pythium aphanidermatum, Pythium irregulare, Pythium ultimum);

### Japanese Radish Disease:

Alternaria leaf spot (Alternaria brassicicola);

### Turfgrass Diseases:

dollar spot (Sclerotinia homoeocarpa), brown patch and large patch (Rhizoctonia solani), and Pythium blight (Pythium aphanidermatum);

### Banana Disease:

Sigatoka disease (Mycosphaerella fijiensis, Mycosphaerella musicola);

### Lentils Disease:

Ascochyta blight (Ascochyta lentis);

### Chickpea Disease:

Ascochyta blight (Ascochyta rabiei);

### Green Pepper Disease:

anthracnose (Colletotrichum scovillei);

### Mango Disease:

anthracnose (Colletotrichum acutatum);

### Fruit Trees Diseases:

white root rot (Rosellinia necatrix), and violet root rot (Helicobasidium mompa);
Postharvest diseases of fruits (for example, apple and pear):
Mucor rot diseases (Mucor piriformis);

Seed diseases or diseases in the early stages of the growth of various plants caused by Aspergillus spp., Penicillium spp., Fusarium spp., Gibberella spp., Tricoderma spp., Thielaviopsis spp., Rhizopus spp., Mucor spp., Corticium spp., Phoma spp., Rhizoctonia spp. or Diplodia spp.; and the like;

### Viral Diseases:

Lettuce big-vein disease transmitted by Olpidium brassicae, and viral diseases of several crops transmitted by Polymixa spp. (e.g. Polymyxa betae and Polymyxa graminis);

### Diseases Caused by Bacteria:

bacterial seedling blight of rice (Burkholderia plantarii), bacterial spot of cucumber (Pseudomonas syringae pv. Lachrymans), bacterial wilt of eggplant (Ralstonia solanacearum), canker of citrus (Xanthomonas citri), bacterial soft rot of Chinese cabbage (Erwinia carotovora), scab of potato (Streptomyces scabiei), Goss's wilt of corn (Clavibacter michiganensis), Pierce's disease of grapes, olive and peach (Xylella fastidiosa), and crown gall of Rosacead plants such as apple, peach, cherries (Agrobacterium tumefaciens).

In the above phytopathogenic microorganisms, variations within the species are not particularly limited. Namely, the phytopathogenic microorganisms also include any microorganisms that decrease a sensitivity (which also referred to as “have a resistance”) to a specific fungicide. The decrease of the sensitivity may be attributed to a mutation at a target site (target site mutations), or may be attributed to any factors other than target site mutation (non-target site mutations). Examples of the target site mutations include those wherein amino acid substitution is/are caused at a protein as target site by a mutation of an open reading frame which corresponds to an amino acid sequence of a protein, and those wherein amino acid as a target site is overexpressed by any mutations such as a deletion of suppressor sequence in promotor region, an amplification of an enhancer sequence, and an increase of the copy number of a gene and the others. Examples of the non-target site mutations include an enhancement of efflux function which exhausts extracellularly a fungicide inflowed in a cell by ABC transporter, MFS transporter and the others. Also, a detoxification of a fungicide by a metabolism is included.

Examples of the above-mentioned specific fungicides include nucleic acid synthesis inhibitors (such as phenylamide fungicides, acylamino acid fungicides, DNA topoisomerase type II fungicides), mitosis and cell division inhibitors (such as MBC fungicides, N-phenylcarbamate fungicides), respiratory inhibitors (such as QoI fungicides, QiI fungicides, SDHI fungicides), inhibitors for amino acid synthesis and protein synthesis (such as anilino pyrimidine fungicides), signal transduction inhibitors (such as phenylpyrrole fungicides, dicarboxyimide fungicides), inhibitors for lipid synthesis and cell membrane synthesis (such as phosphorothioate fungicides, dithiolane inhibitors, aromatic hydrocarbon fungicides, heteroaromatic fungicides, carbamate fungicides), sterol biosynthesis inhibitors (such as DMI fungicides including triazoles, hydroxyanilide fungicides, aminopyrazolinone fungicides), cell wall synthesis inhibitors (such as polyoxin inhibitors, carboxylic amide fungicides), melanin synthesis inhibitors (such as MBI-R fungicides, MBI-D fungicides, MBI-P fungicides), as well as the other fungicides (such as cyanoacetoamide oxime fungicides, phenyl acetamide fungicides).

Examples of the amino acid substitution at target site include the followings.

Cytochrome b: G143A, F129L, G137R, I147V, L275F, Y279C, Y279S, M295L, L299F, A126T, Y132C, C133Y, G137V, G137A, G137S, M139V, T145F, T145R, T145S, T145C, T145L, T145Y, T148M, T148V, T148L, T1481, T1481, N256Y, N256K, N256I, E272D, E272G, E272Q, W273L, W273F, Y274S, Y274F, L275S, L275T or L295F;

Cyp51 protein: A311G, A379G, A381G, A410T, A61V, D107V, D134G, D282E, D411N, E297K, F120L, F219S, F449S, F489L, F495I, G138C/R/S, G312A, G412A, G432S, G434C, G448S, G460D/A, G462A, G464S, G484S, G510C, G54E/K/R/V/W, G54W, H147Y, H303Y, H399P, I145F, 1330T, I381V/A, I471T, I475T, K142R, K143E, K147Q, K175N, K197N, L50S, L98H, M145L, M220K/I/T/V, M288L, N125I, N178S, N22D, N284H, N513K, P216L, P384S, P394L, Q141H, Q88H, R467K, S188N, S208T, S297T, S405F, S5081, S5091, S524T, S52T, S79T, T289A, T440A, T454P, T469S, V101F, V136A/C/G, V490L, Y121F, Y131F/H, Y132F/H/N, Y134F, Y134F, Y136F, Y137F, Y140F/H, Y145F, Y431C, Y459C/D/N/S/P/A, Y461D, Y461D/H/S, Y463D/H/N, Y491H or Y68N;

β-tubulin: H6L/Y, Y50C/N/S, Q134K, A165V, E198A/D/G/K/L/Q/V, F200Y, M257L, F200Y, F167Y, Q73R or L240F;

SdhB: H277R/Y, P225H/F/L/T, N230I, H272L/R/V/Y, H278Y/R, H249L/N/Y, H273Y, N225I/T, T268I/A, I269V, H242R, H257L or T253I;

SdhC: H134R, P80H/L, A85V, S73P, T901, I86F, N88S, H154Y/R, K49E, R64K, N75S, G79R, S135R, N87S, H153R, H146R, I29V, N33T, N34T, T79I/N, W80S, A84V, N86K/S/A, G90R, R151T/S, H152R, I161S, G169D or H151R;

SdhD: H133R, H132R, S89P, G109V, D124E/N, H134R, G138V, D145G, 150F, M114V or D129E;

OS-1(Shk1): E753K, G420D, I365N/R/S, V368F, Q369H/P, N373S, T447S, F267L, L290S, T765R, Q777R, T489I, E599K or G736Y; ERG27: S9G, F26S, P57A, T63I, G170R, V1921, L195F, N196T, A210G, I232M, P238S/A, P250S, P269L, P298A, V309M, A314V, S336C, V365A, E368D, N369D, E375K, A378T, L400F/S, Y408S, F412I/S/V/C, A461S or R496T.

Also, examples of phytopathogenic microorganisms whose sensitivity against fungicides are decreased by overexpressing Cyp51 gene, and host plants therefor include the followings.

Septoria tritici of wheat (Reference literature: Pest Management Science. 2012. 68(7). 1034-1040), Rhynchosporium secalis of barley (Reference literature: Molecular Biology and Evolution. 2014. 31(7). 1793-1802), Phakopsora pachyrhizi of soybean (Reference literature: Pest Management Science. 2014. 70(3). 378-388), Venturia inaequalis of apple (Reference literature: Phytopathology. 2016. 106(6). 562-571), Penicillium digitatum of citrus (Reference literature: Applied and Environmental Microbiology. 2000. 66(8). 3421-3426).

The phytopathogenic microorganism which can be controlled by the composition of the present invention may be a plural of the above-mentioned amino acid substitutions. In this case, a plural of the amino acid substitutions may be the identical proteins or the different proteins. Also they may have the non-target site mutations and the target site mutations. For example, phytopathogenic microorganism which causes an amino acid substitution of G143A, F129L and G137R on cytochrome b; phytopathogenic microorganism which has an amino acid substitution of G143A on cytochrome b and also has an amino acid substitution with A311G on Cyp51; phytopathogenic microorganism which has an amino acid substitution of G143A and F129L on cytochrome b and also has an amino acid with A311G on Cyp51; phytopathogenic microorganism which has an amino acid substitution of G143A and F129L on cytochrome b and has an amino acid substitution of H6L/Y on β-tubulin, and further is overexpressed by Cyp51 gene.

Examples of phytopathogenic microorganism having a target site mutation include the followings.

Alternaria alternata which has an amino acid substitution of G143A on cytochrome b;

Alternaria arborescens which has an amino acid substitution of G143A on cytochrome b;

Alternaria solani which has an amino acid substitution of F129L on cytochrome b;

Alternaria tomato which has an amino acid substitution of G143A on cytochrome b;

Botryotinia fuckeliana which has an amino acid substitution of G143A on cytochrome b;

Glomerella graminicola which has an amino acid substitution of G143A on cytochrome b;

Corynespora cassiicola which has an amino acid substitution of G143A on cytochrome b;

Cercospora beticola which has an amino acid substitution of G143A on cytochrome b;

Cercospora sojina which has an amino acid substitution of G143A on cytochrome b;

Cladsporium carpophilum which has an amino acid substitution of G143A on cytochrome b;

Colletotrichum graminicola which has an amino acid substitution of G143A on cytochrome b;

Glomerella cingulata which has an amino acid substitution of G143A on cytochrome b;

Blumeria graminis f. sp. hordei which has an amino acid substitution of G143A on cytochrome b;

Blumeria graminis f. sp. tritici which has an amino acid substitution of G143A on cytochrome b;

Parastagonospora nodorum which has an amino acid substitution of G143A on cytochrome b;

Monographella nivalis which has an amino acid substitution of G143A on cytochrome b;

Microdochium majus which has an amino acid substitution of G143A on cytochrome b;

Mycosphaerella fijiensis which has an amino acid substitution of G143A on cytochrome b;

Didymella rabiei which has an amino acid substitution of G143A on cytochrome b;

Phakopsora pachyrhizi which has an amino acid substitution of F129L on cytochrome b;

Plasmopara viticola which has an amino acid substitution of F129L or G143A on cytochrome b;

Pleospora allii which has an amino acid substitution of G143A on cytochrome b;

Podosphaera fusca which has an amino acid substitution of G143A on cytochrome b;

Podosphaera xanthii which has an amino acid substitution of G143A on cytochrome b;

Podosphaera xanthii which has an amino acid substitution of G143A on cytochrome b;

Pseudoperonospora cubensis which has an amino acid substitution of G143A on cytochrome b;

Magnaporthe oryzae which has an amino acid substitution of F129L or G143A on cytochrome b;

Pyrenophora teres which has an amino acid substitution of F129L on cytochrome b;

Pyrenophora tritici-repentis which has an amino acid substitution of F129L, G137R or G143A on cytochrome b;

Pythium aphanidermatum which has an amino acid substitution of F129L on cytochrome b;

Thanatephorus cucumeris which has an amino acid substitution of F129L or G143A on cytochrome b;

Ramularia collo-cygni which has an amino acid substitution of G143A on cytochrome b;

Rhynchosporium secalis which has an amino acid substitution of G143A on cytochrome b;

Rhizoctonia solani which has an amino acid substitution of F129L on cytochrome b;

Zymoseptoria tritici which has an amino acid substitution of F129L, G137R or G143A on cytochrome b;

Erysiphe necator which has an amino acid substitution of G143A on cytochrome b;

Venturia inaequalis which has an amino acid substitution of G143A on cytochrome b;

Saccharomyces cerevisiae which has an amino acid substitution of I147V on cytochrome b;

Saccharomyces cerevisiae which has an amino acid substitution of L275F on cytochrome b;

Saccharomyces cerevisiae which has an amino acid substitution of Y279C on cytochrome b;

Saccharomyces cerevisiae which has an amino acid substitution of Y279S on cytochrome b;

Saccharomyces cerevisiae which has an amino acid substitution of M295L on cytochrome b;

Puccinia horiana which has an amino acid substitution of L299F on cytochrome b;

fungi or Oomycota each of which has an amino acid substitution of L299F on cytochrome b;

fungi or Oomycota each of which has an amino acid substitution of A126T on cytochrome b;

fungi or Oomycota each of which has an amino acid substitution of Y132C on cytochrome b;

fungi or Oomycota each of which has an amino acid substitution of C133Y on cytochrome b;

fungi or Oomycota each of which has an amino acid substitution of G137V on cytochrome b;

fungi or Oomycota each of which has an amino acid substitution of G137A on cytochrome b;

fungi or Oomycota each of which has an amino acid substitution of G137S on cytochrome b;

fungi or Oomycota each of which has an amino acid substitution of M139V on cytochrome b;

fungi or Oomycota each of which has an amino acid substitution of T145F on cytochrome b;

fungi or Oomycota each of which has an amino acid substitution of T145R on cytochrome b;

fungi or Oomycota each of which has an amino acid substitution of T145S on cytochrome b;

fungi or Oomycota each of which has an amino acid substitution of T145C on cytochrome b;

fungi or Oomycota each of which has an amino acid substitution of T145L on cytochrome b;

fungi or Oomycota each of which has an amino acid substitution of T145Y on cytochrome b;

fungi or Oomycota each of which has an amino acid substitution of T148M on cytochrome b;

fungi or Oomycota each of which has an amino acid substitution of T148V on cytochrome b;

fungi or Oomycota each of which has an amino acid substitution of T148L on cytochrome b;

fungi or Oomycota each of which has an amino acid substitution of T1481 on cytochrome b;

fungi or Oomycota each of which has an amino acid substitution of T148T on cytochrome b;

fungi or Oomycota each of which has an amino acid substitution of N256Y on cytochrome b;

fungi or Oomycota each of which has an amino acid substitution of N256K on cytochrome b;

fungi or Oomycota each of which has an amino acid substitution of N256I on cytochrome b;

fungi or Oomycota each of which has an amino acid substitution of E272D on cytochrome b;

fungi or Oomycota each of which has an amino acid substitution of E272G on cytochrome b;

fungi or Oomycota each of which has an amino acid substitution of E272Q on cytochrome b;

fungi or Oomycota each of which has an amino acid substitution of W273L on cytochrome b;

fungi or Oomycota each of which has an amino acid substitution of W273F on cytochrome b;

fungi or Oomycota each of which has an amino acid substitution of Y274S on cytochrome b;

fungi or Oomycota each of which has an amino acid substitution of Y274F on cytochrome b;

fungi or Oomycota each of which has an amino acid substitution of L275S on cytochrome b;

fungi or Oomycota each of which has an amino acid substitution of L275T on cytochrome b;

fungi or Oomycota each of which has an amino acid substitution of L295F on cytochrome b;

Ajellomyces capsulatus which has an amino acid substitution of Y136F on Cyp51;

Aspergillus flavus which has an amino acid substitution of Y132N, K197N, D282E, M288L, T469S, H399P, D411N or T454P on Cyp51;

Aspergillus fumigatus which has an amino acid substitution of N22D, S52T, G54E/K/R/V/W, Y68N, Q88H, L98H, V101F, Y121F, N1251, G138C/R/S, Q141H, H147Y, P216L, F219S, M220K/I/T/V, T289A, S297T, P394L, Y431C, G432S, G434C, T440A, G448S, Y491H or F495I on Cyp51;

Aspergillus parasiticus which has an amino acid substitution of G54W on Cyp51;

Candida albicans which has an amino acid substitution of A61V, Y132F/H, K143E, S405F, F449S, G464S, R467K or I471T on Cyp51;

Cercospora beticola which has an amino acid substitution of E297K, I330T or P384S on Cyp51;

Blumeria graminis f. sp. hordei which has an amino acid substitution of Y136F, K147Q or S5091 on Cyp51;

Blumeria graminis f. sp. tritici which has an amino acid substitution of S79T, Y136F, or K175N on Cyp51;

Filobasidiella neoformans which has an amino acid substitution of Y145F or G484S on Cyp51;

Monilinia fructicola which has an amino acid substitution of Y136F on Cyp51;

Mycosphaerella fijiensis which has an amino acid substitution of Y136F, A313G, A381G, Y461D, G462A or Y463D/H/N on Cyp51;

Phakopsora pachyrhizi which has an amino acid substitution of F120L, Y131F/H, K142R, I145F or I475T on Cyp51;

Puccinia triticina which has an amino acid substitution of Y134F on Cyp51;

Pyrenophora teres triticina which has an amino acid substitution of F489L on Cyp51;

Pyrenopeziza brassicae which has an amino acid substitution of 55081 on Cyp51;

Saccharomyces cerevisiae which has an amino acid substitution of Y140F/H on Cyp51;

Zymoseptoria tritici which has an amino acid substitution of L50S, D107V, D134G, V136A/C/G, Y137F, M145L, N178S, S188N, S2081, N284H, H303Y, A311G, G312A, A379G, I381V/Δ, A410T, G412A, Y459C/D/N/S/P/Δ, G460D/Δ, Y461D/H/S, V490L, G510C, N513K or S524T on Cyp51;

Erysiphe necator which has an amino acid substitution of Y136F on Cyp51;

Emericella nidulans which has an amino acid substitution of H6L/Y, Y50N/S, Q134K, A165V, E198D/K/Q, F200Y or M257L on β-tubulin;

Botryotinia fuckeliana which has an amino acid substitution of E198A/G/K/V or F200Y on β-tubulin;

Cochliobolus heterostrophus which has an amino acid substitution of F167Y on β-tubulin;

Cercospora beticola which has an amino acid substitution of F167Y or E198A on β-tubulin;

Gibberella fujikuroi which has an amino acid substitution of Y50N, E198V or F200Y on β-tubulin;

Gibberella zeae which has an amino acid substitution of Y50C, Q73R, F167Y, E198K/L/Q or F200Y on β-tubulin;

Helminthosporium solani which has an amino acid substitution of E198A/Q on β-tubulin;

Hypomyces odoratus which has an amino acid substitution of Y50C on β-tubulin;

Parastagonospora nodorum which has an amino acid substitution of H6Y on β-tubulin;

Monilinia fructicola which has an amino acid substitution of H6Y or E198A/K on β-tubulin;

Monilinia laxa which has an amino acid substitution of H6Y or L240F on β-tubulin;

Microdochium majus which has an amino acid substitution of E198A on β-tubulin;

Mycosphaerella fijiensis which has an amino acid substitution of E198A on β-tubulin;

Neurospora crassa which has an amino acid substitution of F167Y or E198G on β-tubulin;

Penicillium aurantiogriseum which has an amino acid substitution of E198A/K or F200Y on β-tubulin;

Penicillium expansum which has an amino acid substitution of F167Y or E198A/K/V on β-tubulin;

Penicillium italicum which has an amino acid substitution of E198K or F200Y on β-tubulin;

Pyrenopeziza brassicae which has an amino acid substitution of L240F on β-tubulin;

Rhynchosporium secalis which has an amino acid substitution of E198G/K or F200Y on β-tubulin;

Sclerotinia homoeocarpa which has an amino acid substitution of E198A/K on β-tubulin;

Sclerotinia sclerotiorum which has an amino acid substitution of E198A on β-tubulin;

Zymoseptoria tritici which has an amino acid substitution of E198A/G on β-tubulin;

Venturia inaequalis which has an amino acid substitution of E198A/K, F200Y or L240F on β-tubulin;

Alternaria alternata which has an amino acid substitution of H277R/Y on SdhB;

Botryotinia fuckeliana which has an amino acid substitution of P225H/F/L/T, N230I or H272L/R/V/Y on SdhB;

Corynespora cassiicola which has an amino acid substitution of H278Y/R on SdhB;

Stagonosporopsis cucurbitacearum which has an amino acid substitution of H277R/Y on SdhB;

Eurotium oryzae which has an amino acid substitution of H249L/N/Y on SdhB;

Pyrenophora teres which has an amino acid substitution of H277Y on SdhB;

Sclerotinia sclerotiorum which has an amino acid substitution of H273Y on SdhB;

Zymoseptoria tritici which has an amino acid substitution of N225I/T, H273Y, 12681/A or I269V on SdhB;

Erysiphe necator which has an amino acid substitution of H242R on SdhB;

Ustilago maydis which has an amino acid substitution of H257L on SdhB;

Venturia inaequalis which has an amino acid substitution of T253I on SdhB;

Alternaria alternata which has an amino acid substitution of H134R on SdhC;

Botryotinia fuckeliana which has an amino acid substitution of P80H/L or A85V on SdhC;

Corynespora cassiicola which has an amino acid substitution of S73P on SdhC;

Eurotium oryzae which has an amino acid substitution of T901 on SdhC;

Phakopsora pachyrhizi which has an amino acid substitution of I86F, N88S or H154Y/R on SdhC;

Pyrenophora teres which has an amino acid substitution of K49E, R64K, N75S, G79R, H134R or S135R on SdhC;

Ramularia collo-cygni which has an amino acid substitution of N87S, H146R or H153R on SdhC;

Sclerotinia sclerotiorum which has an amino acid substitution of H146R on SdhC;

Zymoseptoria tritici which has an amino acid substitution of I29V, N33T, N34T, T79I/N, W80S, A84V, N86K/S/A, G90R, R151T/S, H152R or I161S on SdhC;

Erysiphe necator which has an amino acid substitution of G169D on SdhC;

Venturia inaequalis which has an amino acid substitution of H151R on SdhC;

Alternaria alternata which has an amino acid substitution of H133R on SdhD;

Alternaria solani which has an amino acid substitution of H133R on SdhD;

Botryotinia fuckeliana which has an amino acid substitution of H132R on SdhD;

Corynespora cassiicola which has an amino acid substitution of S89P or G109V on SdhD;

Eurotium oryzae which has an amino acid substitution of D124E on SdhD;

Pyrenophora teres which has an amino acid substitution of D124E/N, H134R, G138V or D145G on SdhD;

Sclerotinia sclerotiorum which has an amino acid substitution of H132R or D145G on SdhD;

Zymoseptoria tritici which has an amino acid substitution of 150F, M114V or D129E on SdhD;

Phytophthora capsici which has an amino acid substitution of Q1077K or V1109L/M on CesA3;

Phytophthora drechsleri which has an amino acid substitution of V1109L on CesA3;

Phytophthora infestans which has an amino acid substitution of G1105A/V or V1109L on CesA3;

Plasmopara viticola which has an amino acid substitution of G1105S/V on CesA3;

Pseudoperonospora cubensis which has an amino acid substitution of G1105V/W on CesA3;

Alternaria brassicicola which has an amino acid substitution of E753K on OS-1(Shk1);

Alternaria longipes which has an amino acid substitution of G420D on OS-1(Shk1);

Botryotinia fuckeliana which has an amino acid substitution of I365N/R/S, V368F, Q369H/P, N373S or T447S on OS-1(Shk1);

Pleospora allii which has an amino acid substitution of F267L, L290S, T765R or Q777R on OS-1(Shk1);

Sclerotinia sclerotiorum which has an amino acid substitution of T489I, E599K or G736Y on OS-1(Shk1);

Botryotinia fuckeliana which has an amino acid substitution of S9G, F26S, P57A, T63I, G170R, V1921, L195F, N196T, A210G, I232M, P238S/A, P250S, P269L, P298A, V309M, A314V, S336C, V365A, E368D, N369D, E375K, A378T, L400F/S, Y408S, F412I/S/V/C, A461S or R496T on ERG27.

Zymoseptoria tritici means the same genus as Septoria tritici.

Examples of the plants which the composition of the present invention can be used include the following plants.

### Crops:

corn (dent corn, flint corn, flour corn, popcorn, glutinous corn, sweet corn), rices (long grain rice, short grain rice, medium grain rice, Japonica rice, tropical Japonica rice, paddy rice, dry-land rice, floating rice, direct sowing rice, transplanted rice, glutinous rice), wheat (bread wheat (hard wheat, soft wheat, medium wheat, red wheat, white wheat), macaroni wheat, spelt wheat, club wheat, autumn sowing type thereof, spring sowing type thereof), barley (two-row barley (=beer brewing barley), six-row barley), naked barley, pearl barley, autumn sowing type thereof, spring sowing type thereof), rye (autumn sowing type thereof, spring sowing type thereof), triticale (autumn sowing type thereof, spring sowing type thereof), oat (autumn sowing type thereof, spring sowing type thereof), sorghum, cotton (upland cotton, pima cotton), soybean (determinate type soybean, indeterminate soybean, semi-determinate type soybean), groundnut (peanut), common bean (kidney bean), Lima bean, Azuki bean, cowpea bean, mung bean, urad dal bean, runner bean, rice bean, moth bean, tepary bean, broad bean, pea, chickpea, lentil, lupin bean, pigeon pea, alfalfa, buckwheat, sugar beet, rapeseed, canola (autumn sowing type thereof, spring sowing type thereof), sun flower, sugar cane, tobacco and the others;

### Vegetables:

solanaceous vegetables (eggplant, tomato, pimento, pepper, bell pepper and potato, and the others),

cucurbitaceous vegetables (cucumber, pumpkin, zucchini, water melon, melon, and squash, and the others),

cruciferous vegetables (Japanese radish, white turnip, horseradish, kohlrabi, Chinese cabbage, cabbage, leaf mustard, broccoli and cauliflower, and the others),

asteraceous vegetables (burdock, crown daisy, artichoke and lettuce, and the others),

liliaceous vegetables (green onion, onion, garlic and asparagus, and the others),

ammiaceous vegetables (for example, carrot, parsley, celery and parsnip, and the others),

chenopodiaceous vegetables (spinach and Swiss chard, and the others),

lamiaceous vegetables (Perilla frutescens, mint, basil, and lavender, and the others),

strawberry, sweet potato, Dioscorea japonica, colocasia, and the others;

### Fruits:

pomaceous fruits (apple, pear, Japanese pear, Chinese quince and quince, and the others),

stone fleshy fruits (peach, plum, nectarine, Prunus mume, cherry fruit, apricot and prune, and the other),

citrus fruits (Citrus unshiu, orange, lemon, lime and grapefruit, and the other),

nuts (chestnut, walnuts, hazelnuts, almond, pistachio, cashew nuts and macadamia nuts, and the other),

berry fruits (blueberry, cranberry, blackberry and raspberry, and the other),

grape, kaki persimmon, olive, Japanese plum, banana, coffee, date palm, coconuts, and the others;

### Others:

tea, mulberry, flowering plant, roadside trees (for example, ash, birch, dogwood, Eucalyptus, Ginkgo biloba, lilac, maple, Quercus, poplar, Judas tree, Liquidambar formosana, plane tree, zelkova, Japanese arborvitae, fir wood, hemlock, juniper, Pinus, Picea, and Taxus cuspidate), flowers, ornamental foliage plants, sods, and grasses.

The above plants are not limited specifically, as long as they are breeds that are usually cultivated.

The above plants may be also a plant that can be generated by a natural crossbreeding, a plant that can be generated by mutations, an F1 hybrid plant, and a transgenic plant (which is also referred to as a genetically modified crop). In general, these plants have characteristics that are tolerance to herbicides, accumulation of toxic substances against pests (which is also referred to as pest resistance), suppression of sensitivity to diseases (which is also referred to as disease resistance), increase of yield potential, improvement of tolerance to biological and abiotic stress factors, modification of quality of products (for example, increase or decrease of the content of ingredient(s), change of composition, or improvement of storability and processability), and the like.

The method for controlling plant diseases of the present invention (hereinafter, referred to as “control method of the present invention”) is carried out by applying each of an effective amount of the present compound A and the present compound B, to a plant or soil where the plant is grows.

The plants encompasses the whole plant and a particular part of the plant. Examples of the particular part of the plants include stems and leaves, flowers, ears, fruits, trunks, branches, crowns, seeds, bulbs, and seedlings. Bulbs described herein represents discoid stems, corms, rhizomes, tubers, tuberous, seed tubers, and tuberous roots of a plant. In the present control method, the ratio of the applied amounts of the present compound A relative to the applied amounts of the present compound B is within a range of usually 1:0.01 to 1:100, and preferably 1:0.1 to 1:10.

In the control method of the present invention, the present compound A and the present compound B may be applied separately to a plant or soil where the plant grows in the same period, but are usually applied as the present composition in terms of a convenience on applying.

In the control method of the present invention, examples of the method of applying the present compound A and the present compound B include foliage treatment, soil treatment, root treatment, and seed treatment.

The foliage treatment includes, for example, a method of applying the present compounds onto surface of a plant to be grown by a foliar application or a stem application.

The root treatment includes, for example, a method of soaking a whole or a root of the plant into a medicinal solution comprising the present compound A and the present compound B, as well as a method of attaching a solid formulation comprising the present compound A, the present compound B and the solid carrier to a root of the plant.

The soil treatment includes, for example, soil broadcast, soil incorporation, and irrigation of the agent solution to a soil.

The seed treatment includes, for example, an applying of the present composition to a seed of the plant to be prevented from the plant disease. Specific examples of the treatment include spray treatment by spraying a suspension of the present composition in a mist form onto a surface of a seed, smear treatment by applying the wettable powder, the emulsifiable concentrate or the flowable formulation of the present composition with added by small amounts of water or as itself to a seed, immersion treatment by immersing a seed into a solution of the present composition for a certain period of time, film-coating treatment and pellet-coating treatment. Also the present composition can be applied to bulbs of the plant according to a similar method to the above spray treatment and smear treatment.

Each dose of the present compound A and the present compound B in the control method of the present invention may be varied depending on a kind of plant to be treated, a kind or a frequency of an occurrence of a plant disease as a control subject, a dosage form, a treatment period, a treatment method, a treatment site, a climate condition, etc. In case of an application to a foliage of the plant or a soil where the plant is grown, a total amount of the present compound A and the present compound B is usually 1 to 500 g, preferably 2 to 200 g, and more preferably 10 to 100 g, per 1000 m². Also each dose of the present compound A and the present compound B in the treatment for seed is usually 0.001 to 10 g, and preferably 0.01 to 1 g, per 1 kg of seeds.

The emulsifiable concentrate, the wettable powder or the flowable formulation, etc., is usually applied by diluting them with water, and then spreading them. In this case, usually, each concentration of the present compound A and the present compound B contain 0.0005 to 2% by weight, and preferably 0.005 to 1% by weight of the present compound A and the present compound B in total. The dust formulation or the granular formulation, etc., is usually applied as itself without diluting them.

### Examples

Hereinafter, the present invention is explained in more detail by Preparation Examples and Examples of the present compound A, Formulation Examples of the present composition, and Test Examples, however, the present invention should not be limited to only the these Examples.

Firstly, the preparation examples of the present compound A is described.

As used herein, Me represents methyl group, Et represents ethyl group, and Pr represents propyl group.

Examples of the present compound A are shown below.

A compound represented by formula (I):

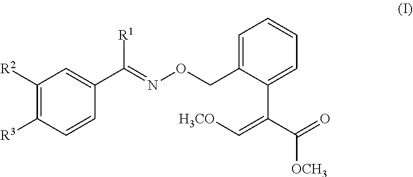

wherein a combination of R¹, R² and R³ represents any combinations indicated in [Table 1] to [Table 3]].

TABLE 1

| Present | | | |
|---|---|---|---|
| compound A | R¹ | R² | R³ |
| A1 | Me | H | H |
| A2 | Me | F | H |
| A3 | Me | F | F |
| A4 | Me | H | F |
| A5 | Me | Cl | H |
| A6 | Me | Cl | Cl |
| A7 | Me | H | Cl |
| A8 | Me | Br | H |
| A9 | Me | Br | Br |
| A10 | Me | H | Br |
| A11 | Me | I | H |
| A12 | Me | I | I |
| A13 | Me | H | I |
| A14 | Me | Me | H |
| A15 | Me | Me | Me |
| A16 | Me | H | Me |
| A17 | Me | Et | H |
| A18 | Me | Et | Et |
| A19 | Me | H | Et |
| A20 | Me | Pr | H |
| A21 | Me | Pr | Pr |
| A22 | Me | H | Pr |
| A23 | Me | CF₃ | H |
| A24 | Me | CF₃ | CF₃ |
| A25 | Me | H | CF₃ |

TABLE 2

| Present | | | |
|---|---|---|---|
| compound A | R¹ | R² | R³ |
| A26 | Me | OMe | H |
| A27 | Me | OMe | OMe |
| A28 | Me | H | OMe |
| A29 | Me | OCF₃ | H |
| A30 | Me | OCF₃ | OCF₃ |
| A31 | Me | H | OCF₃ |
| A32 | Et | H | H |
| A33 | Et | F | H |
| A34 | Et | F | F |
| A35 | Et | H | F |
| A36 | Et | Cl | H |
| A37 | Et | Cl | Cl |
| A38 | Et | H | Cl |
| A39 | Et | Br | H |
| A40 | Et | Br | Br |
| A41 | Et | H | Br |
| A42 | Et | I | H |
| A43 | Et | I | I |
| A44 | Et | H | I |
| A45 | Et | Me | H |
| A46 | Et | Me | Me |
| A47 | Et | H | Me |
| A48 | Et | Et | H |
| A49 | Et | Et | Et |
| A50 | Et | H | Et |

TABLE 3

| Present | | | |
|---|---|---|---|
| compound A | R¹ | R² | R³ |
| A51 | Et | Pr | H |
| A52 | Et | Pr | Pr |
| A53 | Et | H | Pr |
| A54 | Et | CF₃ | H |
| A55 | Et | CF₃ | CF₃ |
| A56 | Et | H | CF₃ |
| A57 | Et | OMe | H |
| A58 | Et | OMe | OMe |
| A59 | Et | H | OMe |
| A60 | Et | OCF₃ | H |
| A61 | Et | OCF₃ | OCF₃ |
| A62 | Et | H | OCF₃ |

A compound represented by formula (II):

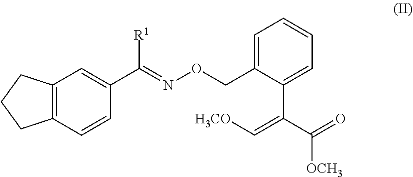

wherein a combination of R¹ represents any combinations indicated in [Table 4].

TABLE 4

| Present | |
|---|---|
| compound A | R¹ |
| A63 | Me |
| A64 | Et |
| A65 | Pr |

A compound represented by formula (III):

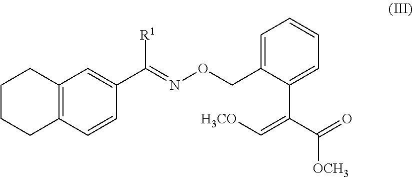

wherein a combination of R¹ represents any combinations indicated in [Table 5].

TABLE 5

| Present compound A | R¹ |
|---|---|
| A66 | Me |
| A67 | Et |
| A68 | Pr |

Specific examples of the present composition are described below. Here the present compound S represents the present compound A1 to the present compound A68.

Composition Class MX1: a composition for controlling plant diseases comprising anyone compound of the present compound S and picoxystrobin within a range of 0.1:1;
Composition Class MX2: a composition for controlling plant diseases comprising anyone compound of the present compound S and picoxystrobin within a range of 1:1;
Composition Class MX3: a composition for controlling plant diseases comprising anyone compound of the present compound S and picoxystrobin within a range of 10:1;
Composition Class MX4: a composition for controlling plant diseases comprising anyone compound of the present compound S and pyraclostrobin within a range of 0.1:1;
Composition Class MX5: a composition for controlling plant diseases comprising anyone compound of the present compound S and pyraclostrobin within a range of 1:1;
Composition Class MX6: a composition for controlling plant diseases comprising anyone compound of the present compound S and pyraclostrobin within a range of 10:1;
Composition Class MX7: a composition for controlling plant diseases comprising anyone compound of the present compound S and metyltetraprole within a range of 0.1:1;
Composition Class MX8: a composition for controlling plant diseases comprising anyone compound of the present compound S and metyltetraprole within a range of 1:1;
Composition Class MX9: a composition for controlling plant diseases comprising anyone compound of the present compound S and metyltetraprole within a range of 10:1;
Composition Class MX10: a composition for controlling plant diseases comprising anyone compound of the present compound S and fenpicoxamid within a range of 0.1:1;
Composition Class MX11: a composition for controlling plant diseases comprising anyone compound of the present compound S and fenpicoxamid within a range of 1:1;
Composition Class MX12: a composition for controlling plant diseases comprising anyone compound of the present compound S and fenpicoxamid within a range of 10:1;
Composition Class MX13: a composition for controlling plant diseases comprising anyone compound of the present compound S and florylpicoxamid within a range of 0.1:1;
Composition Class MX14: a composition for controlling plant diseases comprising anyone compound of the present compound S and florylpicoxamid within a range of 1:1;
Composition Class MX15: a composition for controlling plant diseases comprising anyone compound of the present compound S and florylpicoxamid within a range of 10:1;
Composition Class MX16: a composition for controlling plant diseases comprising anyone compound of the present compound S and fluxapyroxad within a range of 0.1:1;
Composition Class MX17: a composition for controlling plant diseases comprising anyone compound of the present compound S and fluxapyroxad within a range of 1:1;
Composition Class MX18: a composition for controlling plant diseases comprising anyone compound of the present compound S and fluxapyroxad within a range of 10:1;
Composition Class MX19: a composition for controlling plant diseases comprising anyone compound of the present compound S and benzovindiflupyr within a range of 0.1:1;
Composition Class MX20: a composition for controlling plant diseases comprising anyone compound of the present compound S and benzovindiflupyr within a range of 1:1;
Composition Class MX21: a composition for controlling plant diseases comprising anyone compound of the present compound S and benzovindiflupyr within a range of 10:1;
Composition Class MX22: a composition for controlling plant diseases comprising anyone compound of the present compound S and fluindapyr within a range of 0.1:1;
Composition Class MX23: a composition for controlling plant diseases comprising anyone compound of the present compound S and fluindapyr within a range of 1:1;
Composition Class MX24: a composition for controlling plant diseases comprising anyone compound of the present compound S and fluindapyr within a range of 10:1;
Composition Class MX25: a composition for controlling plant diseases comprising anyone compound of the present compound S and pydiflumetofen within a range of 0.1:1;
Composition Class MX26: a composition for controlling plant diseases comprising anyone compound of the present compound S and pydiflumetofen within a range of 1:1;
Composition Class MX27: a composition for controlling plant diseases comprising anyone compound of the present compound S and pydiflumetofen within a range of 10:1;
Composition Class MX28: a composition for controlling plant diseases comprising anyone compound of the present compound S and 3-(difluoromethyl)-N-(2,3-dihydro-1,1,3-trimethyl-1H-inden-4-yl)-1-methyl-1H-pyrazole-4-carboxamide within a range of 0.1:1;
Composition Class MX29: a composition for controlling plant diseases comprising anyone compound of the present compound S and 3-(difluoromethyl)-N-(2,3-dihydro-1,1,3-trimethyl-1H-inden-4-yl)-1-methyl-1H-pyrazole-4-carboxamide within a range of 1:1;
Composition Class MX30: a composition for controlling plant diseases comprising anyone compound of the present compound S and 3-(difluoromethyl)-N-(2,3-dihydro-1,1,3-trimethyl-1H-inden-4-yl)-1-methyl-1H-pyrazole-4-carboxamide within a range of 10:1;
Composition Class MX31: a composition for controlling plant diseases comprising anyone compound of the present compound S and a compound represented by formula (1) within a range of 0.1:1;
Composition Class MX32: a composition for controlling plant diseases comprising anyone compound of the present compound S and a compound represented by formula (1) within a range of 1:1;
Composition Class MX33: a composition for controlling plant diseases comprising anyone compound of the present compound S and a compound represented by formula (1) within a range of 10:1;
Composition Class MX34: a composition for controlling plant diseases comprising anyone compound of the present compound S and a compound represented by formula (2) within a range of 0.1:1;
Composition Class MX35: a composition for controlling plant diseases comprising anyone compound of the present compound S and a compound represented by formula (2) within a range of 1:1;
Composition Class MX36: a composition for controlling plant diseases comprising anyone compound of the present compound S and a compound represented by formula (2) within a range of 10:1;
Composition Class MX37: a composition for controlling plant diseases comprising anyone compound of the present compound S and a compound represented by formula (3) within a range of 0.1:1;
Composition Class MX38: a composition for controlling plant diseases comprising anyone compound of the present compound S and a compound represented by formula (3) within a range of 1:1;
Composition Class MX39: a composition for controlling plant diseases comprising anyone compound of the present compound S and a compound represented by formula (3) within a range of 10:1;
Composition Class MX40: a composition for controlling plant diseases comprising anyone compound of the present compound S and mefentrifluconazole within a range of 0.1:1;
Composition Class MX41: a composition for controlling plant diseases comprising anyone compound of the present compound S and mefentrifluconazole within a range of 1:1;
Composition Class MX42: a composition for controlling plant diseases comprising anyone compound of the present compound S and mefentrifluconazole within a range of 10:1.

Next, the formulation Examples are described. The “parts” represents “part by weight”. Composition MX represents any compositions described in Composition Class MX1 to Composition Class MX42.

### Formulation Example 1

Fifty (50) parts of any one of the composition MX, 3 parts of calcium lignin sulfonate, 2 parts of magnesium lauryl sulfate, and 45 parts of synthetic hydrated silicon dioxide are well mixed-grinding to obtain a formulation.

### Formulation Example 2

Twenty (20) parts of any one of the composition MX, and 1.5 parts of sorbitan trioleate are mixed with 28.5 parts of an aqueous solution containing 2 parts of polyvinyl alcohol, and the mixture is then finely-ground by a wet grinding method. To the mixture is then added 40 parts of an aqueous solution containing 0.05 parts of xanthan gum and 0.1 parts of magnesium aluminum silicate, and 10 parts of propylene glycol is further added thereto, and the mixture is mixed with starring to obtain a formulation.

### Formulation Example 3

Two (2) parts of any one of the composition MX, 88 parts of kaolin clay and 10 parts of talc are mixed-grinding thoroughly to obtain a formulation.

### Formulation Example 4

Five (5) parts of any one of the composition MX, 14 parts of polyoxyethylene styryl phenyl ether, 6 parts of calcium dodecylbenzene sulfonate and 75 parts of xylene are mixed-grinding thoroughly to obtain a formulation.

### Formulation Example 5

Two (2) parts of any one of the composition MX, 1 part of synthetic hydrated silicon dioxide, 2 parts of calcium lignosulfonate, 30 parts of bentonite and 65 parts of kaolin clay are mixed-grinding, and thereto is added water, and the mixture is well kneaded and is then granulated and dried to obtain a formulation.

### Formulation Example 6

Twenty (20) parts of any one of the composition MX, 35 parts of a mixture of white carbon and ammonium polyoxyethylene alkyl ether sulfate (weight ratio is 1:1) and 45 parts of water are well mixed to obtain a formulation.

Next, test Examples are described to show that the present composition is useful for controlling plant diseases.

### Test Example 1: Control Test Against Soybean Rust (Phakopsora pachyrhizi)

Soybean leaf (cv; Kurosengoku) was punched out to 1 cm diameter to prepare a leaf disk. Each 1 mL of an agar medium (agar concentration 1.2%) was dispensed in each well of 24 well microplate. A piece of the leaf disk was placed on agar medium on each well. To a mixture of 1 μL of Sorpol (registered trademark) 1200KX, 4.5 μL of DMSO, and 5 μL of xylene were added 20 μL of a DMSO solution containing a predetermined concentration of any one of the compound selected from the present compound S, and 20 μL of a DMSO solution containing a predetermined concentration of any one of the compound selected from the present compound B, and the solutions were mixed.

The resulting mixture was diluted with ion exchange water to prepare an agent solution containing a predetermined concentration of the test compound. The resulting agent solution was sprayed in 10 μL per one leaf disk. After 1 day, an aqueous suspension of conidia of Phakopsora pachyrhizi (1.0×10⁵/mL) was inoculated onto the leaf disks. After the inoculation, the microplate was placed in a growth chamber (light on for 6 hours, light off for 18 hours, 23° C. temperature, 60% humidity). After 1 day, the leaf disks were air-dried to disappear water droplets on the surface of the leaf disk, and the microplate was placed again in the growth chamber for 12 days (which was “treated area”). Thereafter, a lesion area of soybean rust disease was assessed.

Control effect was calculated by the following “Equation 4” from the ratio of the lesion area of the incidence of disease in the treated area and the ratio of the lesion area of the incidence of disease in the non-treated area.

Control effect=100×(X−Y)/X “Equation 4”

X: Ratio of the lesion area of the incidence of disease in the non-treated area
Y: Ratio of the lesion area of the incidence of disease in the treated area

Here the non-treated area represents an area wherein the same procedures are taken except for using the present compound S and the present compound B.

The results are shown in Table 6 to Table 12.

TABLE 6

| | Conc. | Control |
|---|---|---|
| Composition of the present invention | (ppm) | Effect |
| Present compound A5 + benzovindiflupyr | 10 + 10 | 100 |
| Present compound A5 + benzovindiflupyr | 3 + 30 | 100 |
| Present compound A5 + benzovindiflupyr | 10 + 1 | 100 |
| Present compound A5 + 3-(difluoromethyl)-N-(2,3-dihydro-1,1,3- | 10 + 10 | 100 |
| trimethyl-1H-inden-4-yl)-1-methyl-1H-pyrazole-4-carboxamide | | |
| Present compound A5 + 3-(difluoromethyl)-N-(2,3-dihydro-1,1,3- | 3 + 30 | 100 |
| trimethyl-1H-inden-4-yl)-1-methyl-1H-pyrazole-4-carboxamide | | |
| Present compound A5 + 3-(difluoromethyl)-N-(2,3-dihydro-1,1,3- | 10 + 1 | 100 |
| trimethyl-1H-inden-4-yl)-1-methyl-1H-pyrazole-4-carboxamide | | |
| Present compound A5 + fluindapyr | 10 + 10 | 100 |
| Present compound A5 + fluindapyr | 3 + 30 | 100 |
| Present compound A5 + fluindapyr | 10 + 1 | 100 |
| Present compound A5 + fluxapyroxad | 10 + 10 | 100 |
| Present compound A5 + fluxapyroxad | 3 + 30 | 100 |
| Present compound A5 + fluxapyroxad | 10 + 1 | 100 |
| Present compound A5 + Compound of formula (1) | 10 + 10 | 100 |
| Present compound A5 + Compound of formula (1) | 3 + 30 | 100 |
| Present compound A5 + Compound of formula (1) | 10 + 1 | 100 |
| Present compound A5 + Compound of formula (2) | 10 + 10 | 100 |
| Present compound A5 + Compound of formula (2) | 3 + 30 | 100 |
| Present compound A5 + Compound of formula (2) | 10 + 1 | 100 |
| Present compound A5 + Compound of formula (3) | 10 + 10 | 100 |
| Present compound A5 + Compound of formula (3) | 3 + 30 | 100 |
| Present compound A5 + Compound of formula (3) | 10 + 1 | 100 |
| Present compound A5 + pyraclostrobin | 10 + 10 | 100 |
| Present compound A5 + pyraclostrobin | 5 + 50 | 100 |
| Present compound A5 + pyraclostrobin | 10 + 1 | 100 |
| Present compound A5 + picoxystrobin | 10 + 10 | 100 |
| Present compound A5 + picoxystrobin | 5 + 50 | 100 |
| Present compound A5 + picoxystrobin | 10 + 1 | 100 |
| Present compound A5 + fenpicoxamid | 10 + 10 | 100 |
| Present compound A5 + fenpicoxamid | 5 + 50 | 100 |
| Present compound A5 + fenpicoxamid | 30 + 10 | 100 |
| Present compound A5 + florylpicoxamid | 10 + 10 | 100 |
| Present compound A5 + florylpicoxamid | 5 + 50 | 100 |
| Present compound A5 + florylpicoxamid | 30 + 10 | 100 |

TABLE 7

| | Conc. | Control |
|---|---|---|
| Composition of the present invention | (ppm) | Effect |
| Present compound A7 + benzovindiflupyr | 10 + 10 | 100 |
| Present compound A7 + benzovindiflupyr | 3 + 30 | 100 |
| Present compound A7 + benzovindiflupyr | 10 + 1 | 100 |
| Present compound A7 + 3-(difluoromethyl)-N-(2,3-dihydro-1,1,3- | 10 + 10 | 100 |
| trimethyl-1H-inden-4-yl)-1-methyl-1H-pyrazole-4-carboxamide | | |
| Present compound A7 + 3-(difluoromethyl)-N-(2,3-dihydro-1,1,3- | 3 + 30 | 100 |
| trimethyl-1H-inden-4-yl)-1-methyl-1H-pyrazole-4-carboxamide | | |
| Present compound A7 + 3-(difluoromethyl)-N-(2,3-dihydro-1,1,3- | 10 + 1 | 100 |
| trimethyl-1H-inden-4-yl)-1-methyl-1H-pyrazole-4-carboxamide | | |
| Present compound A7 + fluindapyr | 10 + 10 | 100 |
| Present compound A7 + fluindapyr | 3 + 30 | 100 |
| Present compound A7 + fluindapyr | 10 + 1 | 100 |
| Present compound A7 + fluxapyroxad | 10 + 10 | 100 |
| Present compound A7 + fluxapyroxad | 3 + 30 | 100 |
| Present compound A7 + fluxapyroxad | 10 + 1 | 100 |
| Present compound A7 + Compound of formula (1) | 10 + 10 | 100 |
| Present compound A7 + Compound of formula (1) | 3 + 30 | 100 |
| Present compound A7 + Compound of formula (1) | 10 + 1 | 100 |
| Present compound A7 + Compound of formula (2) | 10 + 10 | 100 |
| Present compound A7 + Compound of formula (2) | 3 + 30 | 100 |
| Present compound A7 + Compound of formula (2) | 10 + 1 | 100 |
| Present compound A7 + Compound of formula (3) | 10 + 10 | 100 |
| Present compound A7 + Compound of formula (3) | 3 + 30 | 100 |
| Present compound A7 + Compound of formula (3) | 10 + 1 | 100 |
| Present compound A7 + pyraclostrobin | 10 + 10 | 100 |
| Present compound A7 + pyraclostrobin | 5 + 50 | 100 |
| Present compound A7 + pyraclostrobin | 10 + 1 | 100 |
| Present compound A7 + picoxystrobin | 10 + 10 | 100 |
| Present compound A7 + picoxystrobin | 5 + 50 | 100 |
| Present compound A7 + picoxystrobin | 10 + 1 | 100 |
| Present compound A7 + fenpicoxamid | 10 + 10 | 100 |
| Present compound A7 + fenpicoxamid | 5 + 50 | 100 |
| Present compound A7 + fenpicoxamid | 30 + 10 | 100 |
| Present compound A7 + florylpicoxamid | 10 + 10 | 100 |
| Present compound A7 + florylpicoxamid | 5 + 50 | 100 |
| Present compound A7 + florylpicoxamid | 30 + 10 | 100 |

TABLE 8

| | Conc. | Control |
|---|---|---|
| Composition of the present invention | (ppm) | Effect |
| Present compound A23 + benzovindiflupyr | 10 + 10 | 100 |
| Present compound A23 + benzovindiflupyr | 3 + 30 | 100 |
| Present compound A23 + benzovindiflupyr | 10 + 1 | 100 |
| Present compound A23 + 3-(difluoromethyl)-N-(2,3-dihydro-1,1,3- | 10 + 10 | 100 |
| trimethyl-1H-inden-4-yl)-1-methyl-1H-pyrazole-4-carboxamide | | |
| Present compound A23 + 3-(difluoromethyl)-N-(2,3-dihydro-1,1,3- | 3 + 30 | 100 |
| trimethyl-1H-inden-4-yl)-1-methyl-1H-pyrazole-4-carboxamide | | |
| Present compound A23 + 3-(difluoromethyl)-N-(2,3-dihydro-1,1,3- | 10 + 1 | 100 |
| trimethyl-1H-inden-4-yl)-1-methyl-1H-pyrazole-4-carboxamide | | |
| Present compound A23 + fluindapyr | 10 + 10 | 100 |
| Present compound A23 + fluindapyr | 3 + 30 | 100 |
| Present compound A23 + fluindapyr | 10 + 1 | 100 |
| Present compound A23 + fluxapyroxad | 10 + 10 | 100 |
| Present compound A23 + fluxapyroxad | 3 + 30 | 100 |
| Present compound A23 + fluxapyroxad | 10 + 1 | 100 |
| Present compound A23 + Compound of formula (1) | 10 + 10 | 100 |
| Present compound A23 + Compound of formula (1) | 3 + 30 | 100 |
| Present compound A23 + Compound of formula (1) | 10 + 1 | 100 |
| Present compound A23 + Compound of formula (2) | 10 + 10 | 100 |
| Present compound A23 + Compound of formula (2) | 3 + 30 | 100 |
| Present compound A23 + Compound of formula (2) | 10 + 1 | 100 |
| Present compound A23 + Compound of formula (3) | 10 + 10 | 100 |
| Present compound A23 + Compound of formula (3) | 3 + 30 | 100 |
| Present compound A23 + Compound of formula (3) | 10 + 1 | 100 |
| Present compound A23 + pyraclostrobin | 10 + 10 | 100 |
| Present compound A23 + pyraclostrobin | 5 + 50 | 100 |
| Present compound A23 + pyraclostrobin | 10 + 1 | 100 |
| Present compound A23 + picoxystrobin | 10 + 10 | 100 |
| Present compound A23 + picoxystrobin | 5 + 50 | 100 |
| Present compound A23 + picoxystrobin | 10 + 1 | 100 |
| Present compound A23 + fenpicoxamid | 10 + 10 | 100 |
| Present compound A23 + fenpicoxamid | 5 + 50 | 100 |
| Present compound A23 + fenpicoxamid | 30 + 10 | 100 |
| Present compound A23 + florylpicoxamid | 10 + 10 | 100 |
| Present compound A23 + florylpicoxamid | 5 + 50 | 100 |
| Present compound A23 + florylpicoxamid | 30 + 10 | 100 |

TABLE 9

| | Conc. | Control |
|---|---|---|
| Composition of the present invention | (ppm) | Effect |
| Present compound A47 + benzovindiflupyr | 10 + 10 | 100 |
| Present compound A47 + benzovindiflupyr | 3 + 30 | 100 |
| Present compound A47 + benzovindiflupyr | 10 + 1 | 100 |
| Present compound A47 + 3-(difluoromethyl)-N-(2,3-dihydro-1,1,3- | 10 + 10 | 100 |
| trimethyl-1H-inden-4-yl)-1-methyl-1H-pyrazole-4-carboxamide | | |
| Present compound A47 + 3-(difluoromethyl)-N-(2,3-dihydro-1,1,3- | 3 + 30 | 100 |
| trimethyl-1H-inden-4-yl)-1-methyl-1H-pyrazole-4-carboxamide | | |
| Present compound A47 + 3-(difluoromethyl)-N-(2,3-dihydro-1,1,3- | 10 + 1 | 100 |
| trimethyl-1H-inden-4-yl)-1-methyl-1H-pyrazole-4-carboxamide | | |
| Present compound A47 + fluindapyr | 10 + 10 | 100 |
| Present compound A47 + fluindapyr | 3 + 30 | 100 |
| Present compound A47 + fluindapyr | 10 + 1 | 100 |
| Present compound A47 + fluxapyroxad | 10 + 10 | 100 |
| Present compound A47 + fluxapyroxad | 3 + 30 | 100 |
| Present compound A47 + fluxapyroxad | 10 + 1 | 100 |
| Present compound A47 + Compound of formula (1) | 10 + 10 | 100 |
| Present compound A47 + Compound of formula (1) | 3 + 30 | 100 |
| Present compound A47 + Compound of formula (1) | 10 + 1 | 100 |
| Present compound A47 + Compound of formula (2) | 10 + 10 | 100 |
| Present compound A47 + Compound of formula (2) | 3 + 30 | 100 |
| Present compound A47 + Compound of formula (2) | 10 + 1 | 100 |
| Present compound A47 + Compound of formula (3) | 10 + 10 | 100 |
| Present compound A47 + Compound of formula (3) | 3 + 30 | 100 |
| Present compound A47 + Compound of formula (3) | 10 + 1 | 100 |
| Present compound A47 + pyraclostrobin | 10 + 10 | 100 |
| Present compound A47 + pyraclostrobin | 5 + 50 | 100 |
| Present compound A47 + pyraclostrobin | 10 + 1 | 100 |
| Present compound A47 + picoxystrobin | 10 + 10 | 100 |
| Present compound A47 + picoxystrobin | 5 + 50 | 100 |
| Present compound A47 + picoxystrobin | 10 + 1 | 100 |
| Present compound A47 + fenpicoxamid | 10 + 10 | 100 |
| Present compound A47 + fenpicoxamid | 5 + 50 | 100 |
| Present compound A47 + fenpicoxamid | 30 + 10 | 100 |
| Present compound A47 + florylpicoxamid | 10 + 10 | 100 |
| Present compound A47 + florylpicoxamid | 5 + 50 | 100 |
| Present compound A47 + florylpicoxamid | 30 + 10 | 100 |

TABLE 10

| | Conc. | Control |
|---|---|---|
| Composition of the present invention | (ppm) | Effect |
| Present compound A56 + benzovindiflupyr | 10 + 10 | 100 |
| Present compound A56 + benzovindiflupyr | 3 + 30 | 100 |
| Present compound A56 + benzovindiflupyr | 10 + 1 | 100 |
| Present compound A56 + 3-(difluoromethyl)-N-(2,3-dihydro-1,1,3- | 10 + 10 | 100 |
| trimethyl-1H-inden-4-yl)-1-methyl-1H-pyrazole-4-carboxamide | | |
| Present compound A56 + 3-(difluoromethyl)-N-(2,3-dihydro-1,1,3- | 3 + 30 | 100 |
| trimethyl-1H-inden-4-yl)-1-methyl-1H-pyrazole-4-carboxamide | | |
| Present compound A56 + 3-(difluoromethyl)-N-(2,3-dihydro-1,1,3- | 10 + 1 | 100 |
| trimethyl-1H-inden-4-yl)-1-methyl-1H-pyrazole-4-carboxamide | | |
| Present compound A56 + fluindapyr | 10 + 10 | 100 |
| Present compound A56 + fluindapyr | 3 + 30 | 100 |
| Present compound A56 + fluindapyr | 10 + 1 | 100 |
| Present compound A56 + fluxapyroxad | 10 + 10 | 100 |
| Present compound A56 + fluxapyroxad | 3 + 30 | 100 |
| Present compound A56 + fluxapyroxad | 10 + 1 | 100 |
| Present compound A56 + Compound of formula (1) | 10 + 10 | 100 |
| Present compound A56 + Compound of formula (1) | 3 + 30 | 100 |
| Present compound A56 + Compound of formula (1) | 10 + 1 | 100 |
| Present compound A56 + Compound of formula (2) | 10 + 10 | 100 |
| Present compound A56 + Compound of formula (2) | 3 + 30 | 100 |
| Present compound A56 + Compound of formula (2) | 10 + 1 | 100 |
| Present compound A56 + Compound of formula (3) | 10 + 10 | 100 |
| Present compound A56 + Compound of formula (3) | 3 + 30 | 100 |
| Present compound A56 + Compound of formula (3) | 10 + 1 | 100 |
| Present compound A56 + pyraclostrobin | 10 + 10 | 100 |
| Present compound A56 + pyraclostrobin | 5 + 50 | 100 |
| Present compound A56 + pyraclostrobin | 10 + 1 | 100 |
| Present compound A56 + picoxystrobin | 10 + 10 | 100 |
| Present compound A56 + picoxystrobin | 5 + 50 | 100 |
| Present compound A56 + picoxystrobin | 10 + 1 | 100 |
| Present compound A56 + fenpicoxamid | 10 + 10 | 100 |
| Present compound A56 + fenpicoxamid | 5 + 50 | 100 |
| Present compound A56 + fenpicoxamid | 30 + 10 | 100 |
| Present compound A56 + florylpicoxamid | 10 + 10 | 100 |
| Present compound A56 + florylpicoxamid | 5 + 50 | 100 |
| Present compound A56 + florylpicoxamid | 30 + 10 | 100 |

TABLE 11

| | Conc. | Control |
|---|---|---|
| Composition of the present invention | (ppm) | Effect |
| Present compound A63 + benzovindiflupyr | 10 + 10 | 100 |
| Present compound A63 + benzovindiflupyr | 3 + 30 | 100 |
| Present compound A63 + benzovindiflupyr | 10 + 1 | 100 |
| Present compound A63 + 3-(difluoromethyl)-N-(2,3-dihydro-1,1,3- | 10 + 10 | 100 |
| trimethyl-1H-inden-4-yl)-1-methyl-1H-pyrazole-4-carboxamide | | |
| Present compound A63 + 3-(difluoromethyl)-N-(2,3-dihydro-1,1,3- | 3 + 30 | 100 |
| trimethyl-1H-inden-4-yl)-1-methyl-1H-pyrazole-4-carboxamide | | |
| Present compound A63 + 3-(difluoromethyl)-N-(2,3-dihydro-1,1,3- | 10 + 1 | 100 |
| trimethyl-1H-inden-4-yl)-1-methyl-1H-pyrazole-4-carboxamide | | |
| Present compound A63 + fluindapyr | 10 + 10 | 100 |
| Present compound A63 + fluindapyr | 3 + 30 | 100 |
| Present compound A63 + fluindapyr | 10 + 1 | 100 |
| Present compound A63 + fluxapyroxad | 10 + 10 | 100 |
| Present compound A63 + fluxapyroxad | 3 + 30 | 100 |
| Present compound A63 + fluxapyroxad | 10 + 1 | 100 |
| Present compound A63 + Compound of formula (1) | 10 + 10 | 100 |
| Present compound A63 + Compound of formula (1) | 3 + 30 | 100 |
| Present compound A63 + Compound of formula (1) | 10 + 1 | 100 |
| Present compound A63 + Compound of formula (2) | 10 + 10 | 100 |
| Present compound A63 + Compound of formula (2) | 3 + 30 | 100 |
| Present compound A63 + Compound of formula (2) | 10 + 1 | 100 |
| Present compound A63 + Compound of formula (3) | 10 + 10 | 100 |
| Present compound A63 + Compound of formula (3) | 3 + 30 | 100 |
| Present compound A63 + Compound of formula (3) | 10 + 1 | 100 |
| Present compound A63 + pyraclostrobin | 10 + 10 | 100 |
| Present compound A63 + pyraclostrobin | 5 + 50 | 100 |
| Present compound A63 + pyraclostrobin | 10 + 1 | 100 |
| Present compound A63 + picoxystrobin | 10 + 10 | 100 |
| Present compound A63 + picoxystrobin | 5 + 50 | 100 |
| Present compound A63 + picoxystrobin | 10 + 1 | 100 |
| Present compound A63 + fenpicoxamid | 10 + 10 | 100 |
| Present compound A63 + fenpicoxamid | 5 + 50 | 100 |
| Present compound A63 + fenpicoxamid | 30 + 10 | 100 |
| Present compound A63 + florylpicoxamid | 10 + 10 | 100 |
| Present compound A63 + florylpicoxamid | 5 + 50 | 100 |
| Present compound A63 + florylpicoxamid | 30 + 10 | 100 |

(Table 12

| | Conc. | Control |
|---|---|---|
| Composition of the present invention | (ppm) | Effect |
| Present compound A66 + benzovindiflupyr | 10 + 10 | 100 |
| Present compound A66 + benzovindiflupyr | 3 + 30 | 100 |
| Present compound A66 + benzovindiflupyr | 10 + 1 | 100 |
| Present compound A66 + 3-(difluoromethyl)-N-(2,3-dihydro-1,1,3- | 10 + 10 | 100 |
| trimethyl-1H-inden-4-yl)-1-methyl-1H-pyrazole-4-carboxamide | | |
| Present compound A66 + 3-(difluoromethyl)-N-(2,3-dihydro-1,1,3- | 3 + 30 | 100 |
| trimethyl-1H-inden-4-yl)-1-methyl-1H-pyrazole-4-carboxamide | | |
| Present compound A66 + 3-(difluoromethyl)-N-(2,3-dihydro-1,1,3- | 10 + 1 | 100 |
| trimethyl-1H-inden-4-yl)-1-methyl-1H-pyrazole-4-carboxamide | | |
| Present compound A66 + fluindapyr | 10 + 10 | 100 |
| Present compound A66 + fluindapyr | 3 + 30 | 100 |
| Present compound A66 + fluindapyr | 10 + 1 | 100 |
| Present compound A66 + fluxapyroxad | 10 + 10 | 100 |
| Present compound A66 + fluxapyroxad | 3 + 30 | 100 |
| Present compound A66 + fluxapyroxad | 10 + 1 | 100 |
| Present compound A66 + Compound of formula (1) | 10 + 10 | 100 |
| Present compound A66 + Compound of formula (1) | 3 + 30 | 100 |
| Present compound A66 + Compound of formula (1) | 10 + 1 | 100 |
| Present compound A66 + Compound of formula (2) | 10 + 10 | 100 |
| Present compound A66 + Compound of formula (2) | 3 + 30 | 100 |
| Present compound A66 + Compound of formula (2) | 10 + 1 | 100 |
| Present compound A66 + Compound of formula (3) | 10 + 10 | 100 |
| Present compound A66 + Compound of formula (3) | 3 + 30 | 100 |
| Present compound A66 + Compound of formula (3) | 10 + 1 | 100 |
| Present compound A66 + pyraclostrobin | 10 + 10 | 100 |
| Present compound A66 + pyraclostrobin | 5 + 50 | 100 |
| Present compound A66 + pyraclostrobin | 10 + 1 | 100 |
| Present compound A66 + picoxystrobin | 10 + 10 | 100 |
| Present compound A66 + picoxystrobin | 5 + 50 | 100 |
| Present compound A66 + picoxystrobin | 10 + 1 | 100 |
| Present compound A66 + fenpicoxamid | 10 + 10 | 100 |
| Present compound A66 + fenpicoxamid | 5 + 50 | 100 |
| Present compound A66 + fenpicoxamid | 30 + 10 | 100 |
| Present compound A66 + florylpicoxamid | 10 + 10 | 100 |
| Present compound A66 + florylpicoxamid | 5 + 50 | 100 |
| Present compound A66 + florylpicoxamid | 30 + 10 | 100 |

### Test Example 2: Control Test Against Wheat Septoria Leaf Blotch Fungus (Septoria tritici)

Each of any one of the compound selected from the present compound S and the present compound B were diluted with DMSO so as to contain a predetermined concentration thereof, and 1 μL of the dilution mixtures were dispensed into titer plate (96 well), and thereafter, thereto was then dispensed 150 μL of a potato dextrose broth (PDB broth) to which conidia of Septoria tritici were inoculated in advance. This plate was cultured at 18° C. for 4 days, thereby allowing Septoria tritici to undergo proliferation, and the absorbance at 550 nm of each well of the titer plate was then measured to determine a degree of growth of Septoria tritici (which was “treated area”).

Control effect was calculated by the following “Equation 5” from the degree of growth.

Control effect=100×(X−Y)/X “Equation 5”

X: Degree of growth of fungus in the non-treated area
Y: Degree of growth of fungus in the treated area

Here the non-treated area represents an area wherein the same procedures are taken except for using the present compound S and the present compound B.

The results are shown in Table 13 to Table 19.

TABLE 13

| | Conc. | Control |
|---|---|---|
| Composition of the present invention | (ppm) | Effect |
| Present compound A5 + pydiflumetofen | 0.04 + 0.04 | 100 |
| Present compound A5 + pydiflumetofen | 0.008 + 0.04 | 100 |
| Present compound A5 + pydiflumetofen | 0.04 + 0.008 | 100 |
| Present compound A5 + mefentrifluconazole | 0.04 + 0.04 | 100 |
| Present compound A5 + mefentrifluconazole | 0.008 + 0.04 | 100 |
| Present compound A5 + mefentrifluconazole | 0.04 + 0.008 | 100 |
| Present compound A5 + metyltetraprole | 0.04 + 0.04 | 100 |
| Present compound A5 + metyltetraprole | 0.008 + 0.04 | 100 |
| Present compound A5 + metyltetraprole | 0.04 + 0.008 | 100 |

TABLE 14

| | Conc. | Control |
|---|---|---|
| Composition of the present invention | (ppm) | Effect |
| Present compound A7 + pydiflumetofen | 0.04 + 0.04 | 100 |
| Present compound A7 + pydiflumetofen | 0.008 + 0.04 | 100 |
| Present compound A7 + pydiflumetofen | 0.04 + 0.008 | 100 |
| Present compound A7 + mefentrifluconazole | 0.04 + 0.04 | 100 |
| Present compound A7 + mefentrifluconazole | 0.008 + 0.04 | 100 |
| Present compound A7 + mefentrifluconazole | 0.04 + 0.008 | 100 |
| Present compound A7 + metyltetraprole | 0.04 + 0.04 | 100 |
| Present compound A7 + metyltetraprole | 0.008 + 0.04 | 100 |
| Present compound A7 + metyltetraprole | 0.04 + 0.008 | 100 |

TABLE 15

| | Conc. | Control |
|---|---|---|
| Composition of the present invention | (ppm) | Effect |
| Present compound A23 + pydiflumetofen | 0.04 + 0.04 | 100 |
| Present compound A23 + pydiflumetofen | 0.008 + 0.04 | 100 |
| Present compound A23 + pydiflumetofen | 0.04 + 0.008 | 100 |
| Present compound A23 + mefentrifluconazole | 0.04 + 0.04 | 100 |
| Present compound A23 + mefentrifluconazole | 0.008 + 0.04 | 100 |
| Present compound A23 + mefentrifluconazole | 0.04 + 0.008 | 100 |
| Present compound A23 + metyltetraprole | 0.04 + 0.04 | 100 |
| Present compound A23 + metyltetraprole | 0.008 + 0.04 | 100 |
| Present compound A23 + metyltetraprole | 0.04 + 0.008 | 100 |

TABLE 16

| | Conc. | Control |
|---|---|---|
| Composition of the present invention | (ppm) | Effect |
| Present compound A47 + pydiflumetofen | 0.04 + 0.04 | 100 |
| Present compound A47 + pydiflumetofen | 0.008 + 0.04 | 100 |
| Present compound A47 + pydiflumetofen | 0.04 + 0.008 | 100 |
| Present compound A47 + mefentrifluconazole | 0.04 + 0.04 | 100 |
| Present compound A47 + mefentrifluconazole | 0.008 + 0.04 | 100 |
| Present compound A47 + mefentrifluconazole | 0.04 + 0.008 | 100 |
| Present compound A47 + metyltetraprole | 0.04 + 0.04 | 100 |
| Present compound A47 + metyltetraprole | 0.008 + 0.04 | 100 |
| Present compound A47 + metyltetraprole | 0.04 + 0.008 | 100 |

TABLE 17

| | Conc. | Control |
|---|---|---|
| Composition of the present invention | (ppm) | Effect |
| Present compound A56 + pydiflumetofen | 0.04 + 0.04 | 100 |
| Present compound A56 + pydiflumetofen | 0.008 + 0.04 | 100 |
| Present compound A56 + pydiflumetofen | 0.04 + 0.008 | 100 |
| Present compound A56 + mefentrifluconazole | 0.04 + 0.04 | 100 |
| Present compound A56 + mefentrifluconazole | 0.008 + 0.04 | 100 |
| Present compound A56 + mefentrifluconazole | 0.04 + 0.008 | 100 |
| Present compound A56 + metyltetraprole | 0.04 + 0.04 | 100 |
| Present compound A56 + metyltetraprole | 0.008 + 0.04 | 100 |
| Present compound A56 + metyltetraprole | 0.04 + 0.008 | 100 |

TABLE 18

| | Conc. | Control |
|---|---|---|
| Composition of the present invention | (ppm) | Effect |
| Present compound A63 + pydiflumetofen | 0.04 + 0.04 | 100 |
| Present compound A63 + pydiflumetofen | 0.008 + 0.04 | 100 |
| Present compound A63 + pydiflumetofen | 0.04 + 0.008 | 100 |
| Present compound A63 + mefentrifluconazole | 0.04 + 0.04 | 100 |
| Present compound A63 + mefentrifluconazole | 0.008 + 0.04 | 100 |
| Present compound A63 + mefentrifluconazole | 0.04 + 0.008 | 100 |
| Present compound A63 + metyltetraprole | 0.04 + 0.04 | 100 |
| Present compound A63 + metyltetraprole | 0.008 + 0.04 | 100 |
| Present compound A63 + metyltetraprole | 0.04 + 0.008 | 100 |

TABLE 19

| | Conc. | Control |
|---|---|---|
| Composition of the present invention | (ppm) | Effect |
| Present compound A66 + pydiflumetofen | 0.04 + 0.04 | 100 |
| Present compound A66 + pydiflumetofen | 0.008 + 0.04 | 100 |
| Present compound A66 + pydiflumetofen | 0.04 + 0.008 | 100 |
| Present compound A66 + mefentrifluconazole | 0.04 + 0.04 | 100 |
| Present compound A66 + mefentrifluconazole | 0.008 + 0.04 | 100 |
| Present compound A66 + mefentrifluconazole | 0.04 + 0.008 | 100 |
| Present compound A66 + metyltetraprole | 0.04 + 0.04 | 100 |
| Present compound A66 + metyltetraprole | 0.008 + 0.04 | 100 |
| Present compound A66 + metyltetraprole | 0.04 + 0.008 | 100 |

### INDUSTRIAL APPLICABILITY

The composition for controlling plant diseases of the present invention can control plant diseases.

1. A composition for controlling a plant disease comprising a compound represented by the following formula (I) and one or more compounds selected from Group (B);
a formula (I): 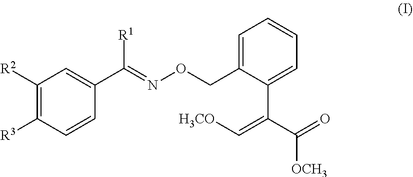
wherein
R¹ represents a C1-C3 alkyl group,
R² and R³ are identical to or different from each other and represent a hydrogen atom, a halogen atom, a C1-C3 alkyl group optionally having one or more halogen atoms, or a C1-C3 alkoxy group optionally having one or more halogen atoms, or
R² and R³ may combine with each other to form —CH₂CH₂CH₂—, or —CH₂CH₂CH₂CH₂—;
Group (B): a group consisting of the following sub-groups (B-1), (B-2) and (B-3),
Sub-group (B-1): a mitochondrial electron transport chain complex III inhibitor, which is selected from
a group consisting of picoxystrobin, pyraclostrobin, metyltetraprole, fenpicoxamid, and florylpicoxamid,
Sub-group (B-2): a mitochondrial electron transport chain complex II inhibitor, which is selected from
a group consisting of fluxapyroxad, benzovindiflupyr, fluindapyr, pydiflumetofen, 3-(difluoromethyl)-N-(2,3-dihydro-1,1,3-trimethyl-1H-inden-4-yl)-1-methyl-1H-pyrazole-4-carboxamide, a compound represented by formula (1), a compound represented by formula (2), and a compound represented by formula (3), 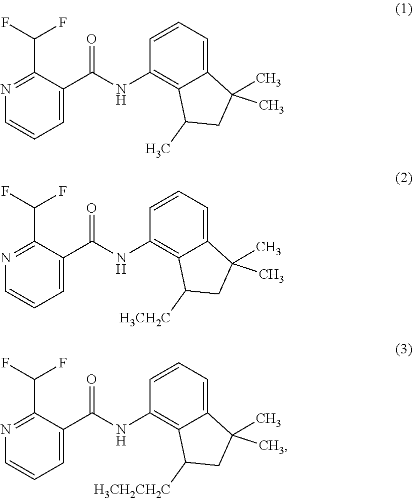
and
Sub-group (B-3): a sterol biosynthesis inhibitor, which is
mefentrifluconazole.

2. The composition for controlling a plant disease according to claim 1, wherein the compound represented by formula (I) is a compound of formula (I) wherein R² represents a hydrogen atom, R³ represents a C1-C3 alkyl group optionally having one or more halogen atoms, a C1-C3 alkoxy group optionally having one or more halogen atoms, or a halogen atom.

3. The composition for controlling a plant disease according to claim 1, wherein the compound represented by formula (I) is a compound of formula (I) wherein R³ represents a hydrogen atom, R² represents a C1-C3 alkyl group optionally having one or more halogen atoms, a C1-C3 alkoxy group optionally having one or more halogen atoms, or a halogen atom.

4. The composition for controlling a plant disease according to claim 1, wherein the compound represented by formula (I) is
methyl(2E)-2-(2-{[({(1E)-1-[5,6,7,8-tetrahydronaphthalen-2-yl]ethylidene}amino)oxy]methyl}phenyl)-3-methoxy-2-propenoate;
methyl(2E)-2-(2-{[({(1E)-1-[2,3-dihydro-1H-inden-5-yl]ethylidene}amino)oxy]methyl}phenyl)-3-methoxy-2-propenoate;
methyl(2E)-2-(2-{[({(1E)-1-[3-chlorophenyl]ethylidene}amino)oxy]methyl}phenyl)-3-methoxy-2-propenoate;
methyl(2E)-2-(2-{[({(1E)-1-[4-chlorophenyl]ethylidene}amino)oxy]methyl}phenyl)-3-methoxy-2-propenoate;
methyl(2E)-2-(2-{[({(1E)-1-[3-(trifluoromethyl)phenyl]ethylidene}amino)oxy]methyl}phenyl)-3-methoxy-2-propenoate;
methyl(2E)-2-{2-[({[(1E)-1-(4-methylphenyl)propylidene]amino}oxy)methyl]phenyl}-3-methoxy-2-propenoate; or
methyl(2E)-2-(2-{[({(1E)-1-[4-(trifluoromethyl)phenyl]propylidene}amino)oxy]methyl}phenyl)-3-methoxy-2-propenoate.

5. The composition for controlling a plant disease according to claim 1, wherein
in Group (B),
Sub-group (B-1) is a group selected from a group consisting of metyltetraprole, fenpicoxamid, and florylpicoxamid,
Sub-group (B-2) is selected from a group consisting of pydiflumetofen, 3-(difluoromethyl)-N-(2,3-dihydro-1,1,3-trimethyl-1H-inden-4-yl)-1-methyl-1H-pyrazole-4-carboxamide, a compound represented by formula (1), a compound represented by formula (2), and a compound represented by formula (3): 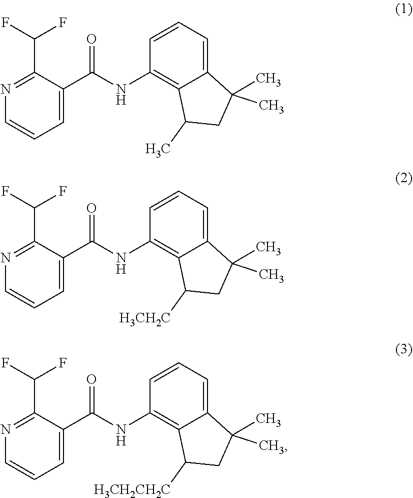
and
Sub-group (B-3) is mefentrifluconazole.

6. The composition for controlling a plant disease according to claim 1, wherein a weight ratio of the compound represented by formula (I) and one or more of the compounds selected from Group (B) is within a range of 1:0.01 to 1:100.

7. The composition for controlling a plant disease according to claim 1, wherein a weight ratio of the compound represented by formula (I) and one or more of the compounds selected from Group (B) is within a range of 1:0.1 to 1:10.

8. A method for controlling a plant disease which comprises applying an effective amount of the composition for controlling a plant disease according to claim 1 to a plant or soil where a plant grows.

9. (canceled)
